(19) United States
(12) Patent Application Publication
(10) Pub. No.: US 2019/0209568 A1
(43) Pub. Date: Jul. 11, 2019

(54) METHOD OF PREVENTING OR TREATING HEARING LOSS

(71) Applicant: SUPPORT-VENTURE GMBH, Basel (CH)

(72) Inventor: Alexander BAUSCH, Riehen (CH)

(21) Appl. No.: 16/325,686

(22) PCT Filed: Aug. 15, 2017
(86) PCT No.: PCT/EP2017/070683

§ 371 Date: Feb. 14, 2019

(30) Foreign Application Priority Data
Aug. 17, 2016 (EP) 16184541.7

(51) Int. Cl. A61K 31/519; A61K 31/4439; A61K 9/00; A61P 27/16

(52) U.S. Cl. CPC A61K 31/519; A61K 31/4439; A61P 27/16; A61K 9/0046; A61K 9/0019

(57) ABSTRACT

The present invention relates to methods of preventing or treating hearing loss and methods of preventing or inhibiting hair cell degeneration or hair cell death in a subject.

### THE FIELD OF THE INVENTION

The present invention relates to methods of preventing or treating hearing loss and methods of preventing or inhibiting hair cell degeneration or hair cell death in a subject.

### BACKGROUND OF THE INVENTION

Hearing loss is related to damage of auditory hair cells e.g. apoptosis of the hair cells as a consequence of e.g. a continuous stress situation or a traumatic event e.g. leading to the activation of inflammatory pathways. Hearing loss can be caused e.g. by a noise trauma, by a medical intervention, by ischemic injury, by a non specific stress leading to sudden hearing loss or by age or can be chemically induced, wherein the chemical induction is caused e.g. by an antibiotic or a chemotherapeutic agent. Child hearing loss might be caused by pre or post natal deficiencies in the energy homeostasis of auditory cells. Hearing loss can also be caused by mitochondrial dysfunction. (C. M. Sue PhD, FRACP1, Cochlear origin of hearing loss in MELAS syndrome, Annals of Neurology. Volume 43, Issue 3, pages 350-359, March 1998). In addition a link between metabolic syndrome and hearing loss could be shown (Barrenas M L, Jonsson B, Tuvemo T, Hellstrom P A, Lundgren M, J Clin Endocrinol Metab. 2005 August; 90(8):4452-6. Epub 2005 May 31). Hearing loss can be of sensorineural origin caused by a damage leading to malnutrition of the cells in early brain development.

Hair cells are fully differentiated and are not replaced after cell death (only a few thousand cells from birth). It is well described in the literature that after stress and damage of the hair cells, the cells can enter in a resting state with no functionality related to the hearing process but remain viable. Approaches to stimulate development or regeneration of new hair cells e.g. by administering growth factors or by stem cell-based therapies in order to achieve disease modification bear the risk of pro-tumorigenic side-effects.

Hearing impairment is a major global health issue with profound societal and economic impact affecting over 275 million people world-wide. The occurrence of hearing loss is rapidly rising, due to e.g. increasing noise exposure and aging populations. With no approved pharmaceutical therapies available today, the unmet medical need is very high. In particular there is a need for providing effective methods for prevention and subsequent treatment of hearing loss which allow for immediate as well as long term maintenance of preventive and/or therapeutic effects.

### SUMMARY OF THE INVENTION

The present invention relates generally to methods of preventing or treating hearing loss and methods of preventing or inhibiting hair cell degeneration or hair cell death using a pharmaceutical combination comprising a PPAR agonist, such as e.g. pioglitazone and a p38 inhibitor, e.g. compound of formula I or II, such as e.g. pamapimod.

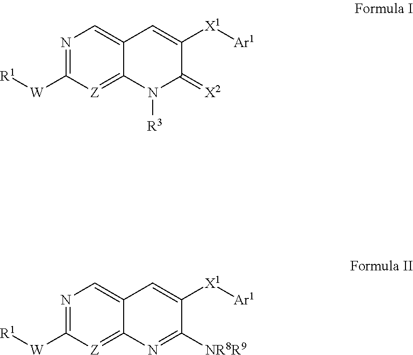

The present invention provides methods which allow for protection of hair cells from stress, e.g. from noise induced stress, from surgery induced stress or from chemically induced stress, such as stress induced by an antibiotic or by a chemotherapeutic agent or from unspecific stress which may cause hearing loss. Using the methods described herein, immediate and subsequent long term maintenance of preventive and/or therapeutic effect can be achieved. In a standard model established in hearing loss research, it could be shown that treatment with a pharmaceutical combination comprising a PPAR agonist and a p38 inhibitor, e.g. a compound of formula I or II protects hair cells, of which upon exposure to an antibiotic 50% are normally destroyed within 24 hours. While the addition of each agent alone gave partial protection, the addition of the pharmaceutical combination of the invention surprisingly and unexpectedly was able to give full protection to hair cells and prevented hair cells treated with antibiotic from apoptosis and cell death.

Accordingly, in a first aspect, the present invention provides a pharmaceutical combination comprising:

(a) a PPAR agonist;
(b) a p38 kinase inhibitor; and optionally
(c) one or more pharmaceutically acceptable diluents, excipients or carriers
for use in a method of preventing or treating hearing loss in a subject.

In a further aspect, the present invention provides a pharmaceutical combination comprising:

(a) a PPAR agonist;
(b) a p38 kinase inhibitor; and optionally
(c) one or more pharmaceutically acceptable diluents, excipients or carriers
for use in a method of preventing or inhibiting hair cell degeneration or hair cell death in a subject.

In a further aspect, the present invention provides a pharmaceutical combination comprising:

(a) a PPAR agonist;
(b) a compound of formula I or II

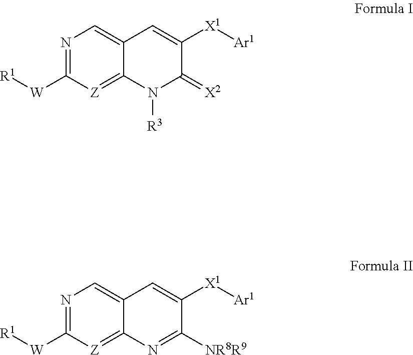

or pharmaceutically acceptable salts thereof, wherein
Z is N or CH;
W is NR²;
X¹ is O, NR⁴ (where R⁴ is hydrogen or alkyl), S, or CR⁵R⁶ (where R⁵ and R⁶ are independently hydrogen or alkyl) or C═O;
X² is O or NR⁷;
Ar¹ is aryl or heteroaryl;
R² is hydrogen, alkyl, acyl, alkoxycarbonyl, aryloxycarbonyl, heteroalkylcarbonyl, heteroalkyloxycarbonyl or —R²¹-R²² where R²¹ is alkylene or —C(═O)— and R²² is alkyl or alkoxy;
R¹ is hydrogen, alkyl, haloalkyl, aryl, aralkyl, heteroaryl, heteroaralkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl, R¹²—SO₂-heterocycloamino (where R¹² is haloalkyl, aryl, aralkyl, heteroaryl or heteroaralkyl), —Y¹—C(O)—Y²—R¹¹ (where Y¹ and Y² are independently either absent or an alkylene group and R¹¹ is hydrogen, alkyl, haloalkyl, hydroxy, alkoxy, amino, monoalkylamino or dialkylamino), (heterocyclyl)(cycloalkyl)alkyl or (heterocyclyl)(heteroaryl)alkyl;
R³ is hydrogen, alkyl, cycloalkyl, cycloalkylalkyl, aryl, aralkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino), amino, monoalkylamino, dialkylamino or NR³²—Y³—R³³ (where Y³ is —C(O), —C(O)O—, —C(O)NR³⁴, S(O)₂ or S(O)₂NR³⁵; R³², R³⁴ and R³⁵ are independently hydrogen or alkyl; and R³³ is hydrogen, alkyl, cycloalkyl, cycloalkylalkyl, heteroalkyl or optionally substituted phenyl) or acyl;
R⁷ is hydrogen or alkyl; and
R⁸ and R⁹ are independently hydrogen, alkyl, aryl, aralkyl, cycloalkyl, cycloalkylalkyl, heteroalkyl, alkylsulfonyl, arylsulfonyl, —C(O)—R⁸¹ (where R⁸¹ is alkyl, aryl, aralkyl, cycloalkyl, cycloalkylalkyl, heteroalkyl, alkoxy, aryloxy, amino, mono- or di-alkylamino, arylamino or aryl(alkyl)amino) or R⁸ and R⁹ together form ═CR⁸²R⁸³ (where R⁸² and R⁸³ are independently hydrogen, alkyl, cycloalkyl, cycloalkylalkyl or optionally substituted phenyl);
   and optionally
   (c) one or more pharmaceutically acceptable diluents, excipients or carriers
   for use in a method of preventing or treating hearing loss in a subject.

In a further aspect, the present invention provides a pharmaceutical combination comprising:

(a) a PPAR agonist;
(b) a compound of the formula I or II

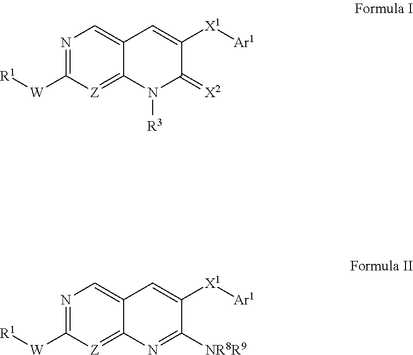

or pharmaceutically acceptable salts thereof, wherein
Z is N or CH;
W is NR²;
X¹ is O, NR⁴ (where R⁴ is hydrogen or alkyl), S, or CR⁵R⁶ (where R⁵ and R⁶ are independently hydrogen or alkyl) or C═O;
X² is O or NR⁷;
Ar¹ is aryl or heteroaryl;
R² is hydrogen, alkyl, acyl, alkoxycarbonyl, aryloxycarbonyl, heteroalkylcarbonyl, heteroalkyloxycarbonyl or —R²¹-R²² where R²¹ is alkylene or —C(═O)— and R²² is alkyl or alkoxy;
R¹ is hydrogen, alkyl, haloalkyl, aryl, aralkyl, heteroaryl, heteroaralkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl, R¹²—SO₂-heterocycloamino (where R¹² is haloalkyl, aryl, aralkyl, heteroaryl or heteroaralkyl), —Y¹—C(O)—Y²—R¹¹ (where Y¹ and Y² are independently either absent or an alkylene group and R¹¹ is hydrogen, alkyl, haloalkyl, hydroxy, alkoxy, amino, monoalkylamino or dialkylamino), (heterocyclyl)(cycloalkyl)alkyl or (heterocyclyl)(heteroaryl)alkyl;
R³ is hydrogen, alkyl, cycloalkyl, cycloalkylalkyl, aryl, aralkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino), amino, monoalkylamino, dialkylamino or NR³²—Y³—R³³ (where Y³ is —C(O), —C(O)O—, —C(O)NR³⁴, S(O)₂ or S(O)₂NR³⁵; R³², R³⁴ and R³⁵ are independently hydrogen or alkyl; and R³³ is hydrogen, alkyl, cycloalkyl, cycloalkylalkyl, heteroalkyl or optionally substituted phenyl) or acyl;
R⁷ is hydrogen or alkyl; and
R⁸ and R⁹ are independently hydrogen, alkyl, aryl, aralkyl, cycloalkyl, cycloalkylalkyl, heteroalkyl, alkylsulfonyl, arylsulfonyl, —C(O)—R^{8′} (where R⁸¹ is alkyl, aryl, aralkyl, cycloalkyl, cycloalkylalkyl, heteroalkyl, alkoxy, aryloxy, amino, mono- or di-alkylamino, arylamino or aryl(alkyl)amino) or R⁸ and R⁹ together form ═CR⁸²R⁸³ (where R⁸² and R⁸³ are independently hydrogen, alkyl, cycloalkyl, cycloalkylalkyl or optionally substituted phenyl);
   and optionally
   (c) one or more pharmaceutically acceptable diluents, excipients or carriers
   for use in a method of preventing or inhibiting hair cell degeneration or hair cell death in a subject.

In a further aspect, the present invention relates to a kit for preventing or treating hearing loss or preventing and/or for inhibiting hair cell degeneration or hair cell death in a subject, comprising a pharmaceutical combination of the invention.

### BRIEF DESCRIPTION OF THE FIGURES

FIG. 1 shows the average number of auditory hair cells remaining in the basal turn of the organ of Corti (OC) following gentamicin treatment with or without pioglitazone or pamapimod. Gentamicin (50 μM) treatment resulted in reduction of hair cell number by approximately 50%. Pioglitazone at 5 μM or pamapimod at 30 μM partially protected hair cells. In each condition, only approximately 20% of hair cells were lost. In contrast, the combination of pioglitazone 5 μM+pamapimod 30 μM resulted in preservation of 100% of the hair cells. The values were averaged for the 10 OCs used for each condition. Significant differences between treatment groups were determined using analysis of variance (ANOVA) followed by the least significant difference (LSD) post-hoc test (Stat View 5.0). Differences associated with P-values of less than 0.05 were considered statistically significant. The data are presented as the mean±SD.

FIG. 2 shows the average number of auditory hair cells remaining in the basal turn of the organ of Corti (OC) following gentamicin treatment with or without pioglitazone or pamapimod. Gentamicin (100 μM) treatment resulted in reduction of hair cell number by approximately 50%. Treatment with the single agents (1 μM pioglitazone or 1 μM pamapimod) partially prevented the loss of hair cells with pioglitazone providing approximately 40% protection and pamapimod providing approximately 30% protection. Surprisingly and unexpectedly, the combination of 1 μM pioglitazone and 1 μM pamapimod provided approximately 100% protection, demonstrating a synergistic effect. The values were averaged for the 10 OCs used for each condition. Significant differences between treatment groups were determined using analysis of variance (ANOVA) followed by the least significant difference (LSD) post-hoc test (Stat View 5.0). Differences associated with P-values of less than 0.05 were considered statistically significant. The data are presented as the mean±SD.

### DETAILED DESCRIPTION OF THE INVENTION

The present invention provides methods of preventing or treating hearing loss and methods of preventing or inhibiting hair cell degeneration or hair cell death.

For the purposes of interpreting this specification, the following definitions will apply and whenever appropriate, terms used in the singular will also include the plural and vice versa. It is to be understood that the terminology used herein is for the purpose of describing particular embodiments only and is not intended to be limiting.

The terms “comprising”, “having”, and “including” are to be construed as open-ended terms (i.e., meaning “including, but not limited to”) unless otherwise noted.

The term “pharmaceutically acceptable carrier” as used herein refers to a carrier or excipient or diluent that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The term “individual,” “subject” or “patient” are used herein interchangeably. In certain embodiments, the subject is a mammal. Mammals include, but are not limited to primates (including human and non-human primates). In a preferred embodiment, the subject is a human.

The term “about” as used herein refers to +/−10% of a given measurement.

Particle size is generally reported on a cumulative distribution by volume basis. The term “micronized” as used herein means particles with a median particle size, expressed by using the cumulative distribution by volume, of between about 1 μm and about 75 μm. Preferably 50% of the particles (Dv50) of the micronized PPAR agonist as referred herein are smaller than or equal to about about 50 μm, more preferably smaller than or equal to about 10 μm, most preferably smaller than or equal to about 5 μm, in particular about 1 to about 10 μm, more particular about about 2 to about 8 μm, even more particular about 3 to about 5 μm, most particular about 4 μm. Micronization is a process of reducing the average diameter of particles of a solid material, whereby the particles are mostly passed through a jet mill. Other mill types may be used as well, e.g. pin mills. The required particle size specification may as well be achieved without a specific milling step by appropriate process conditions in the final precipitation step of the drug substance chemical production. Size reduction is used to increase the surface area of a drug substance and thereby modulate formulation dissolution properties. Micronization is also used to maintain a narrow and consistent particle size distribution for any formulation described herein.

A further purpose of micronization is to allow an easy application of the formulations of the invention by a parenteral syringe. In some embodiments, the needle is wider than a 18 gauge needle. In another embodiment, the needle gauge is from 18 gauge to 30 gauge. In a further embodiment, the needle is a 21 gauge needle. Depending upon the thickness or viscosity of a composition disclosed herein, the gauge level of the syringe or hypodermic needle are varied accordingly. Thus, the formulations of the invention comprising micronized PPAR agonists are ejected e.g. from a 1 mL syringe adapted with a 50 mm length 21 G needle (nominal inner diameter 0.495 mm) without any plugging or clogging.

### PPAR Agonists

The term “PPAR agonist” as used herein refers to a drug that is activating peroxisome proliferator activated receptor (PPAR) such as PPAR gamma receptor, PPAR alpha receptor, PPAR delta receptor or combinations thereof and includes PPAR gamma agonists such as e.g. pioglitazone, troglitazone or rosiglitazone, PPAR alpha agonists such as e.g. fibrates such as fenofibrate (fenofibric acid), clofibrate or gemfibrozil, PPAR dual agonists (PPAR alpha/gamma or PPAR alpha/delta agonists) such as e.g. aleglitazar, muraglitazar, tesaglitazar, ragaglitazar, saroglitazar, GFT505 or naveglitazar, PPAR delta agonists such as e.g. GW501516, PPAR pan agonists (PPAR alpha/delta/gamma agonist) or selective PPAR modulators such as e.g. INT131 and the pharmaceutically acceptable salts of these compounds. Usually PPAR gamma agonists, PPAR modulators, PPAR alpha agonists and/or PPAR alpha/gamma dual agonists are used in the pharmaceutical combinations of the present invention, in particular PPAR gamma agonists, PPAR alpha agonists and/or PPAR alpha/gamma dual agonists are used in the pharmaceutical combinations of the present invention, more particularly PPAR gamma agonists selected from the group consisting of pioglitazone, rosiglitazone, troglitazone and pharmaceutically acceptable salts thereof, preferably pioglitazone or pharmaceutically acceptable salts thereof. PPAR alpha agonists used in the pharmaceutical combinations of the present invention are selected from the group consisting of fenofibrate (fenofibric acid), clofibrate, gemfibrozil and pharmaceutically acceptable salts thereof, preferably fenofibrate (fenofibric acid) or pharmaceutically acceptable salts thereof. PPAR alpha/gamma dual agonists used in the pharmaceutical combinations of the present invention are selected from the group consisting of aleglitazar, muraglitazar, tesaglitazar, ragaglitazar, saroglitazar, GFT505, naveglitazar or pharmaceutically acceptable salts thereof, preferably muraglitazar, tesaglitazar or pharmaceutically acceptable salts thereof. Preferably PPAR gamma agonists are used in the pharmaceutical combinations of the present invention, more preferably PPAR gamma agonists or modulators selected from the group consisting of pioglitazone, rosiglitazone, troglitazone, INT131 and pharmaceutically acceptable salts thereof, even more preferably PPAR gamma agonists selected from the group consisting of pioglitazone, rosiglitazone, troglitazone and pharmaceutically acceptable salts thereof are used. Even more preferably, pioglitazone or a pharmaceutically acceptable salt thereof, in particular pioglitazone hydrochloride is used in the pharmaceutical combinations of the present invention. Most preferably, micronized pioglitazone hydrochloride is used. Thus in a preferred embodiment, a micronized PPAR agonist is used in the pharmaceutical combinations of the invention. In one embodiment, a thiazolidinedione PPAR agonist is used in the pharmaceutical combinations of the invention. Suitable thiazolidinedione PPAR agonists are for example pioglitazone, troglitazone, rosiglitazone or pharmaceutically acceptable salts thereof. A particularly suitable thiazolidinone PPAR agonist is pioglitazone or a pharmaceutically acceptable salt thereof, in particular pioglitazone hydrochloride.

Pioglitazone is described e.g. in U.S. Pat. No. 4,687,777 or in Dormandy J A, Charbonnel B, Eckland D J, Erdmann E, Massi-Benedetti M, Moules I K, Skene A M, Tan M H, Lefèbvre P J, Murray G D, Standl E, Wilcox R G, Wilhelmsen L, Betteridge J, Birkeland K, Golay A, Heine R J, Korányi L, Laakso M, Mokan M, Norkus A, Pirags V, Podar T, Scheen A, Scherbaum W, Schernthaner G, Schmitz O, Skrha J, Smith U, Taton J; PROactive investigators. Lancet. 2005 Oct. 8; 366(9493):1279-89, and is represented by the structural formula indicated below:

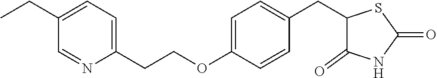 [SMILES: CCc1ccc(CCOc2ccc(CC3SC(=O)NC3=O)cc2)nc1]

Troglitazone is described e.g. in Florez J C, Jablonski K A, Sun M W, Bayley N, Kahn S E, Shamoon H, Hamman R F, Knowler W C, Nathan D M, Altshuler D; Diabetes Prevention Program Research Group. J Clin Endocrinol Metab. 2007 April; 92(4):1502-9 and is represented by the structural formula indicated below:

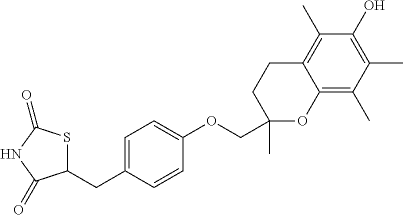 [SMILES: Cc1c(C)c2c(c(C)c1O)CCC(C)(COc1ccc(CC3SC(=O)NC3=O)cc1)O2]

Rosiglitazone is described e.g. in Nissen S E, Wolski K. N Engl J Med. 2007 Jun. 14; 356(24):2457-71. Erratum in: N Engl J Med. 2007 Jul. 5; 357(1):100. Fenofibrate is described e.g. in Bonds D E, Craven T E, Buse J, Crouse J R, Cuddihy R, Elam M, Ginsberg H N, Kirchner K, Marcovina S, Mychaleckyj J C, O'Connor P J, Sperl-Hillen J A. Diabetologia. 2012 June; 55(6):1641-50 and is represented by the structural formula indicated below:

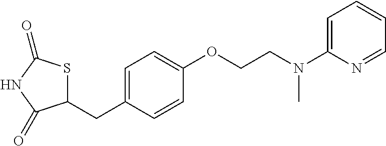 [SMILES: CN(CCOc1ccc(CC2SC(=O)NC2=O)cc1)c1ccccn1]

Clofibrate is described e.g. in Rabkin S W, Hayden M, Frohlich J. Atherosclerosis. 1988 October; 73(2-3):233-40 and is represented by the structural formula indicated below:

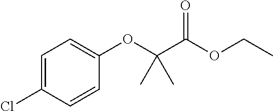 [SMILES: CCOC(=O)C(C)(C)Oc1ccc(Cl)cc1]

Fenofibrate (fenofibric acid) is described e.g. in Schima S M, Maciejewski S R, Hilleman D E, Williams M A, Mohiuddin S M. Expert Opin Pharmacother. 2010 April; 11(5):731-8 and is represented by the structural formula indicated below:

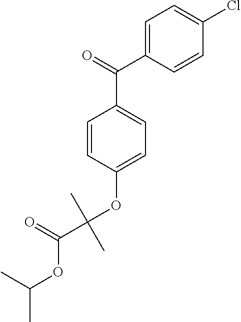 [SMILES: CC(C)OC(=O)C(C)(C)Oc1ccc(C(=O)c2ccc(Cl)cc2)cc1]

Gemfibrozil is described e.g. in Adabag A S, Mithani S, Al Aloul B, Collins D, Bertog S, Bloomfield H E; Veterans Affairs High-Density Lipoprotein Cholesterol Intervention Trial Study Group. Am Heart J. 2009 May; 157(5):913-8 and is represented by the structural formula indicated below:

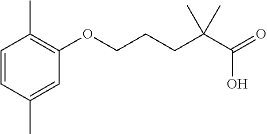 [SMILES: Cc1ccc(C)c(OCCCC(C)(C)C(=O)O)c1]

Aleglitazar is described e.g. in Lincoff A M, Tardif J C, Schwartz G G, Nicholls S J, Rydén L, Neal B, Malmberg K, Wedel H, Buse J B, Henry R R, Weichert A, Cannata R, Svensson A, Volz D, Grobbee D E; AleCardio Investigators. JAMA. 2014 Apr. 16; 311(15):1515-25 and is represented by the structural formula indicated below:

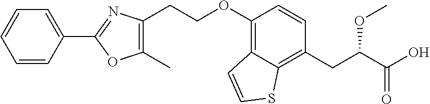 [SMILES: CO[C@@H](Cc1ccc(OCCc2nc(-c3ccccc3)oc2C)c2ccsc12)C(=O)O]

Muraglitazar is described e.g. in Fernandez M, Gastaldelli A, Triplitt C, Hardies J, Casolaro A, Petz R, Tantiwong P, Musi N, Cersosimo E, Ferrannini E, DeFronzo R A. Diabetes Obes Metab. 2011 October; 13(10):893-902 and is represented by the structural formula indicated below:

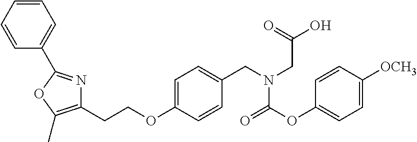 [SMILES: COc1ccc(OC(=O)N(CC(=O)O)Cc2ccc(OCCc3nc(-c4ccccc4)oc3C)cc2)cc1]

Tesaglitazar is described e.g. in Bays H, McElhattan J, Bryzinski B S; GALLANT 6 Study Group. Diab Vasc Dis Res. 2007 September; 4(3):181-93 and is represented by the structural formula indicated below:

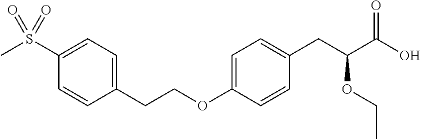 [SMILES: CCO[C@@H](Cc1ccc(OCCc2ccc(S(C)(=O)=O)cc2)cc1)C(=O)O]

Ragaglitazar is described e.g. in Saad M F, Greco S, Osei K, Lewin A J, Edwards C, Nunez M, Reinhardt R R; Ragaglitazar Dose-Ranging Study Group. Diabetes Care. 2004 June; 27(6):1324-9 and is represented by the structural formula indicated below:

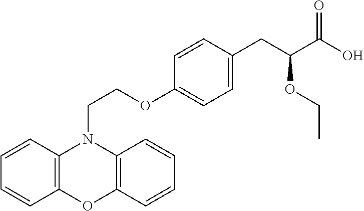 [SMILES: CCO[C@@H](Cc1ccc(OCCN2c3ccccc3Oc3ccccc32)cc1)C(=O)O]

Saroglitazar is described e.g. in Agrawal R. Curr Drug Targets. 2014 February; 15(2): 151-5. and is represented by the structural formula indicated below:

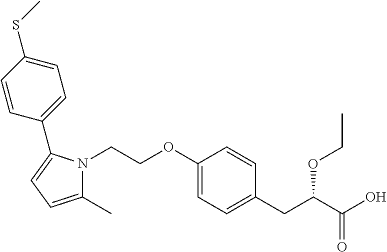 [SMILES: CCO[C@@H](Cc1ccc(OCCn2c(C)ccc2-c2ccc(SC)cc2)cc1)C(=O)O]

Naveglitazar is described e.g. in Ahlawat P, Srinivas N R. Eur J Drug Metab Pharmacokinet. 2008 July-September; 33(3):187-90. GW501516 is described e.g. in Wang X, Sng M K, Foo S, Chong H C, Lee W L, Tang M B, Ng K W, Luo B, Choong C, Wong M T, Tong B M, Chiba S, Loo S C, Zhu P, Tan N S. J Control Release. 2015 Jan. 10; 197:138-47 and is represented by the structural formula indicated below:

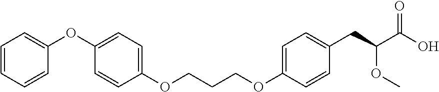 [SMILES: CO[C@@H](Cc1ccc(OCCCOc2ccc(Oc3ccccc3)cc2)cc1)C(=O)O]

GFT505 is described e.g. in Cariou B, Staels B. Expert Opin Investig Drugs. 2014 October; 23(10): 1441-8 and is represented by the structural formula indicated below:

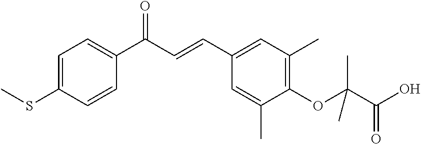 [SMILES: CSc1ccc(C(=O)/C=C/c2cc(C)c(OC(C)(C)C(=O)O)c(C)c2)cc1]

INT131 is described e.g. in. Taygerly J P, McGee L R, Rubenstein S M, Houze J B, Cushing T D, Li Y, Motani A, Chen J L, Frankmoelle W, Ye G, Learned M R, Jaen J, Miao S, Timmermans P B, Thoolen M, Keamey P, Flygare J, Beckmann H, Weiszmann J, Lindstrom M, Walker N, Liu J, Biermann D, Wang Z, Hagiwara A, Iida T, Aramaki H, Kitao Y, Shinkai H, Furukawa N, Nishiu J, Nakamura M. Bioorg Med Chem. 2013 Feb. 15; 21(4):979-92 and is represented by the structural formula indicated below:

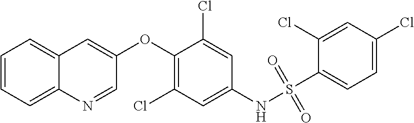 [SMILES: O=S(=O)(Nc1cc(Cl)c(Oc2cnc3ccccc3c2)c(Cl)c1)c1ccc(Cl)cc1Cl]

PPAR activation by the PPAR agonist is usually strong in the low nanomolar range to low micromolar range, e.g in a range of 0.1 nM to 100 μM. In some embodiments the PPAR activation is weak or partial, i.e. a PPAR agonist is used in the methods of the present invention which yields maximal activation of PPAR-receptor in a reporter assay system of 10% to 100% compared to a reference PPAR agonist which is known to causes a maximum PPAR activation. The preferred target for interaction of the PPAR agonist is the hair cell, which is most preferred, neural cells, and endothelial cells, and further includes adipocytes, hepatocytes, immune cells such as e.g. macrophages or dendritic cells, or skeletal muscle cells.

### p38 Kinase Inhibitors

The term “p38 kinase inhibitor” or “p38 inhibitor” which are both used interchangeably herein refers to a drug that is inhibiting a p38 mitogen-activated protein (MAP) kinase, such as p38-α (MAPK14), p38-β (MAPK11), p38-γ (MAPK12/ERK6), and/or p38-δ (MAPK13/SAPK4). In a preferred embodiment, a micronized p38 inhibitor is used in the pharmaceutical combination of the invention. Examples of p38 inhibitors include a compound of formula I or II as defined above. Further examples of p38 inhibitors include pamapimod, losmapimod, dilmapimod, AZD7624, ARRY-371797, LY2228820, R9111, PH-797804, BIRB 796, VX-702, VX-745, SB 239063, SB202190, SCIO 469, BMS 582949 and pharmaceutically acceptable salts thereof.

Thus, in a preferred embodiment, the p38 inhibitor for use in a pharmaceutical combination according to the invention is selected from the group consisting of pamapimod, losmapimod, dilmapimod, AZD7624, ARRY-371797, LY2228820, R9111, PH-797804, BIRB 796, VX-702, VX-745, SB 239063, SB202190, SCIO 469, and BMS 582949 or a pharmaceutically acceptable salt thereof, in particular selected from the group consisting of pamapimod, losmapimod, dilmapimod, ARRY-371797, LY2228820, R9111, PH-797804, BIRB 796, VX-702, VX-745 SB 239063, SB202190, SCIO 469, and BMS 582949 or a pharmaceutically acceptable salt thereof.

In a particularly preferred embodiment, the p38 inhibitor is pamapimod, having the chemical name 6-(2,4-Difluorophenoxy)-2-[3-hydroxy-1-(2-hydroxyethyl)-propylamino]-8-methyl-8H-pyrido[2,3-d]pyrimidin-7-one and the chemical formula III or a pharmaceutically acceptable salt thereof.

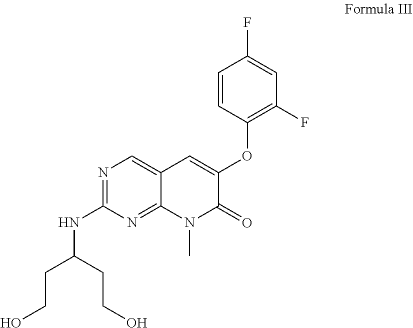 [SMILES: Cn1c(=O)c(Oc2ccc(F)cc2F)cc2cnc(NC(CCO)CCO)nc21]

A further particularly preferred p38 inhibitor is R9111, having the chemical name 6-(2,4-Difluorophenoxy)-2-[(S)-2-hydroxy-1-methyl-ethylamino]-8-[(S)-2-hydroxy-propyl]-8H-pyrido[2,3-d]pyrimidin-7-one and the chemical formula IV or a pharmaceutically acceptable salt thereof.

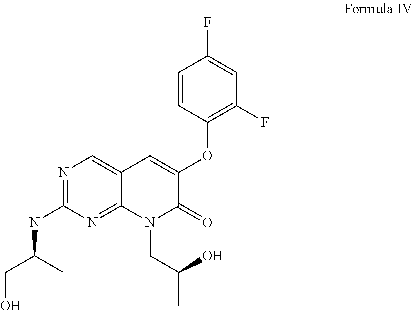 [SMILES: C[C@H](O)Cn1c(=O)c(Oc2ccc(F)cc2F)cc2cnc(N[C@@H](C)CO)nc21]

Pamapimod and its synthesis are described e.g. in WO2008/151992 and in WO2002/064594 and in e.g. Hill R J, Dabbagh K, Phippard D, Li C, Suttmann R T, Welch M, Papp E, Song K W, Chang K C, Leaffer D, Kim Y-N, Roberts R T, Zabka T S, Aud D, Dal Porto J, Manning A M, Peng S L, Goldstein D M, and Wong B R; Pamapimod, a Novel p38 Mitogen-Activated Protein Kinase Inhibitor: Preclinical Analysis of Efficacy and Selectivity J Pharmacol Exp Ther. December 2008 327:610-619.

Losmapimod is described in e.g. Cheriyan J, Webb A J, Sarov-Blat L, Elkhawad M, Wallace S M, Mäki-Petäjä KM, Collier D J, Morgan J, Fang Z, Willette R N, Lepore J J, Cockcroft J R, Sprecher D L, Wilkinson I B. Inhibition of p38 mitogen-activated protein kinase improves nitric oxide-mediated vasodilatation and reduces inflammation in hypercholesterolemia. Circulation. 2011 Feb. 8; 123(5):515-23, and is represented by the structural formula indicated below:

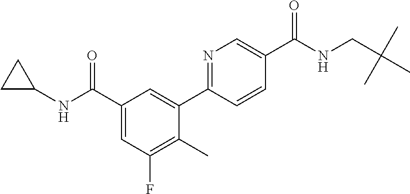 [SMILES: Cc1c(F)cc(C(=O)NC2CC2)cc1-c1ccc(C(=O)NCC(C)(C)C)cn1]

Dilmapimod is described in e.g. Christie J D, Vaslef S, Chang P K, May A K, Gunn S R, Yang S, Hardes K, Kahl L, Powley W M, Lipson D A, Bayliffe A I, Lazaar A L. A Randomized Dose-Escalation Study of the Safety and Anti-Inflammatory Activity of the p38 Mitogen-Activated Protein Kinase Inhibitor Dilmapimod in Severe Trauma Subjects at Risk for Acute Respiratory Distress Syndrome. Crit Care Med. 2015 September; 43(9):1859-69, and is represented by the structural formula indicated below:

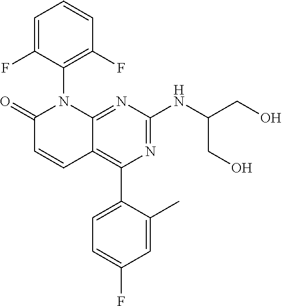 [SMILES: Cc1cc(F)ccc1-c1nc(NC(CO)CO)nc2c1ccc(=O)n2-c1c(F)cccc1F]

LY2228820 is described in e.g. Campbell R M, Anderson B D, Brooks N A, Brooks H B, Chan E M, De Dios A, Gilmour R, Graff J R, Jambrina E, Mader M, McCann D, Na S, Parsons S H, Pratt S E, Shih C, Stancato L F, Starling J J, Tate C, Velasco J A, Wang Y, Ye X S. Characterization of LY2228820 dimesylate, a potent and selective inhibitor of p38 MAPK with antitumor activity. Mol Cancer Ther. 2014 February; 13(2):364-74, and is represented by the structural formula indicated below:

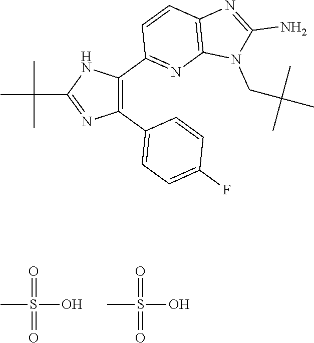

AZD7624 is described in PatelN, Cunoosamy D, Hegelund-Myrback T, Pehrson R, Taib Z, Jansson P, Lundin S, Greenaway S, Clarke G, Siew L. AZD7624, an inhaled p38 inhibitor for COPD, attenuates lung and systemic inflammation after LPS Challenge in humans. Eur Resp J. DOI: 10.1183/13993003.1 September 2015.

ARRY-371797 is described in e.g. Muchir A, Wu W, Choi J C, Iwata S, Morrow J, Homma S, Worman H J. Abnormal p38α mitogen-activated protein kinase signaling in dilated cardiomyopathy caused by lamin A/C gene mutation. Hum Mol Genet. 2012 Oct. 1; 21(19):4325-33 and is represented by the structural formula indicated below:

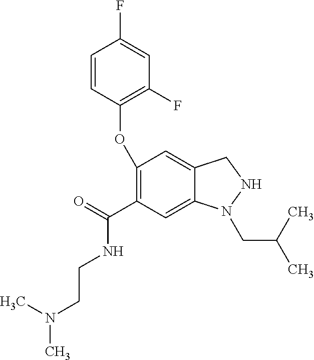 [SMILES: CC(C)CN1NCc2cc(Oc3ccc(F)cc3F)c(C(=O)NCCN(C)C)cc21]

R9111 and its synthesis is described in WO2005/047284 and in e.g. Hill R J, Dabbagh K, Phippard D, Li C, Suttmann R T, Welch M, Papp E, Song K W, Chang K C, Leaffer D, Kim Y-N, Roberts R T, Zabka T S, Aud D, Dal Porto J, Manning A M, Peng S L, Goldstein D M, and Wong B R; Pamapimod, a Novel p38 Mitogen-Activated Protein Kinase Inhibitor: Preclinical Analysis of Efficacy and Selectivity J Pharmacol Exp Ther. December 2008 327:610-619.

PH-797804 is described in e.g. Xing L, Devadas B, Devraj R V, Selness S R, Shieh H, Walker J K, Mao M, Messing D, Samas B, Yang J Z, Anderson G D, Webb E G, Monahan J B. Discovery and characterization of atropisomer PH-797804, a p38 MAP kinase inhibitor, as a clinical drug candidate. ChemMedChem. 2012 Feb. 6; 7(2):273-80, and is represented by the structural formula indicated below:

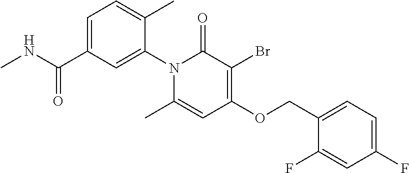 [SMILES: CNC(=O)c1ccc(C)c(-n2c(C)cc(OCc3ccc(F)cc3F)c(Br)c2=O)c1]

BIRB 796 is described in e.g. Dietrich J, Hulme C, Hurley L H. The design, synthesis, and evaluation of 8 hybrid DFG-out allosteric kinase inhibitors: a structural analysis of the binding interactions of Gleevec, Nexavar, and BIRB-796. Bioorg Med Chem. 2010 Aug. 1; 18(15):5738-48, and is represented by the structural formula indicated below:

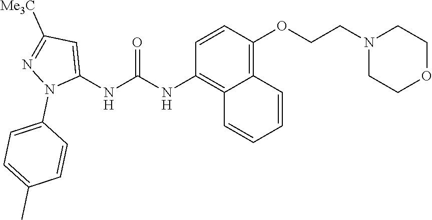

VX-702 is described in e.g. Damjanov N, Kauffman R S, Spencer-Green G T.

Efficacy, pharmacodynamics, and safety of VX-702, a novel p38 MAPK inhibitor, in rheumatoid arthritis: results of two randomized, double-blind, placebo-controlled clinical studies. Arthritis Rheum. 2009 May; 60(5):1232-41, and is represented by the structural formula indicated below:

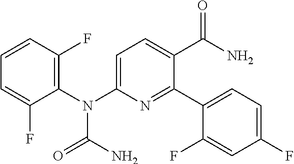 [SMILES: NC(=O)c1ccc(N(C(N)=O)c2c(F)cccc2F)nc1-c1ccc(F)cc1F]

VX-745 is described in e.g. Duffy J P, Harrington E M, Salituro F G, Cochran J E, Green J, Gao H, Bemis G W, Evindar G, Galullo V P, Ford P J, Germann U A, Wilson K P, Bellon S F, Chen G, Taslimi P, Jones P, Huang C, Pazhanisamy S, Wang Y M, Murcko M A, Su M S. The Discovery of VX-745: A Novel and Selective p38α Kinase Inhibitor. ACS Med Chem Lett. 2011 Jul. 28; 2(10):758-63, and is represented by the structural formula indicated below:

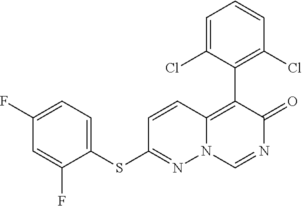 [SMILES: O=c1ncn2nc(Sc3ccc(F)cc3F)ccc2c1-c1c(Cl)cccc1Cl]

SB239063 is described in e.g. Strassburger M, Braun H, Reymann K G. Anti-inflammatory treatment with the p38 mitogen-activated protein kinase inhibitor SB239063 is neuroprotective, decreases the number of activated microglia and facilitates neurogenesis in oxygen-glucose-deprived hippocampal slice cultures. Eur J Pharmacol. 2008 Sep. 11; 592(1-3):55-61, and is represented by the structural formula indicated below:

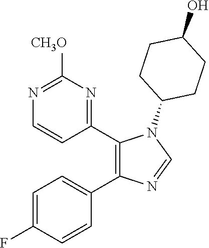

SB202190 is described in e.g. Hirosawa M, Nakahara M, Otosaka R, Imoto A, Okazaki T, Takahashi S. The p38 pathway inhibitor SB202190 activates MEK/MAPK to stimulate the growth of leukemia cells. Leuk Res. 2009 May; 33(5):693-9, and is represented by the structural formula indicated below:

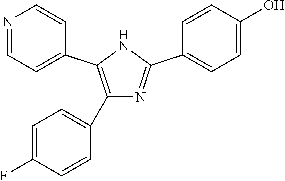 [SMILES: Oc1ccc(-c2nc(-c3ccc(F)cc3)c(-c3ccncc3)[nH]2)cc1]

SCIO469 is described in e.g. Sokol L, Cripe L, Kantarjian H, Sekeres M A, Parmar S, Greenberg P, Goldberg S L, Bhushan V, Shammo J, Hohl R, Verma A, Garcia-Manero G, Li Y P, Lowe A, Zhu J, List A F. Randomized, dose-escalation study of the p38α MAPK inhibitor SCIO-469 in patients with myelodysplastic syndrome. Leukemia. 2013 April; 27(4):977-80, and is represented by the structural formula indicated below:

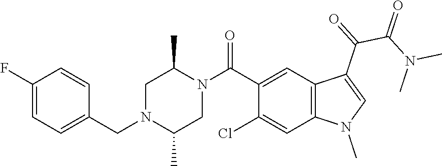 [SMILES: C[C@@H]1CN(Cc2ccc(F)cc2)[C@@H](C)CN1C(=O)c1cc2c(C(=O)C(=O)N(C)C)cn(C)c2cc1Cl]

BMS 582949 is described in e.g. Liu C, Lin J, Wrobleski S T, Lin S, Hynes J, Wu H, Dyckman A J, Li T, Wityak J, Gillooly K M, Pitt S, Shen D R, Zhang R F, McIntyre K W, Salter-Cid L, Shuster D J, Zhang H, Marathe P H, Doweyko A M, Sack J S, Kiefer S E, Kish K F, Newitt J A, McKinnon M, Dodd J H, Barrish J C, Schieven G L, Leftheris K. Discovery of 4-(5-(cyclopropylcarbamoyl)-2-methylphenylamino)-5-methyl-N-propylpyrrolo[1,2-f][1,2,4]triazine-6-carboxamide (BMS-582949), a clinical p38a MAP kinase inhibitor for the treatment of inflammatory diseases. J Med Chem. 2010 Sep. 23; 53(18):6629-39, and is represented by the structural formula indicated below:

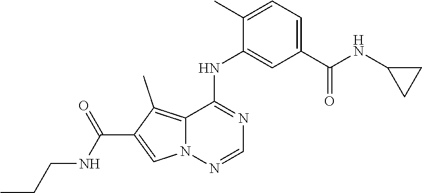 [SMILES: CCCNC(=O)c1cn2ncnc(Nc3cc(C(=O)NC4CC4)ccc3C)c2c1C]

### Compounds of Formulae I and II

In one embodiment, the p38 inhibitor for use in a pharmaceutical combination according to the invention is a compound of formula I

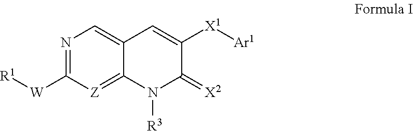

or pharmaceutically acceptable salts thereof, wherein
Z is N or CH;
W is NR²;
X¹ is O, NR⁴ (where R⁴ is hydrogen or alkyl), S, or CR⁵R⁶ (where R⁵ and R⁶ are independently hydrogen or alkyl) or C═O;
X² is O or NR⁷;
Ar¹ is aryl or heteroaryl;
R² is hydrogen, alkyl, acyl, alkoxycarbonyl, aryloxycarbonyl, heteroalkylcarbonyl, heteroalkyloxycarbonyl or —R²¹-R²² where R²¹ is alkylene or —C(═O)— and R²² is alkyl or alkoxy;
R¹ is hydrogen, alkyl, haloalkyl, aryl, aralkyl, heteroaryl, heteroaralkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl, R¹²—SO₂-heterocycloamino (where R¹² is haloalkyl, aryl, aralkyl, heteroaryl or heteroaralkyl), —Y¹—C(O)—Y²—R¹¹ (where Y¹ and Y² are independently either absent or an alkylene group and R¹¹ is hydrogen, alkyl, haloalkyl, hydroxy, alkoxy, amino, monoalkylamino or dialkylamino), (heterocyclyl)(cycloalkyl)alkyl or (heterocyclyl)(heteroaryl)alkyl;
R³ is hydrogen, alkyl, cycloalkyl, cycloalkylalkyl, aryl, aralkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino), amino, monoalkylamino, dialkylamino or NR³²—Y³—R³³ (where Y³ is —C(O), —C(O)O—, —C(O)NR³⁴, S(O)₂ or S(O)₂NR³⁵; R³², R³⁴ and R³⁵ are independently hydrogen or alkyl; and R³³ is hydrogen, alkyl, cycloalkyl, cycloalkylalkyl, heteroalkyl or optionally substituted phenyl) or acyl; and
R⁷ is hydrogen or alkyl
   and optionally one or more pharmaceutically acceptable diluents, excipients or carriers.

In a further embodiment, the p38 inhibitor for use in a pharmaceutical combination according to the invention is a compound of formula I wherein X¹ is NR⁴ and X² is NR⁷ or X¹ and X² are each O, wherein R⁴ and R⁷ are as defined above.

In a further embodiment, the p38 inhibitor for use in a pharmaceutical combination according to the invention is a compound of formula I wherein X¹ is NR⁴ or O and X² is NR⁷ or O, wherein R⁴ and R⁷ are as defined above.

In a further embodiment, the p38 inhibitor for use in a pharmaceutical combination according to the invention is a compound of formula I wherein W is NR², wherein R² is hydrogen, alkyl, heteroalkyl, acyl or alkoxycarbonyl, preferably hydrogen or alkyl, more preferably hydrogen.

In yet a further embodiment, the p38 inhibitor for use in a pharmaceutical combination according to the invention is a compound of formula I wherein R¹ is hydrogen, alkyl, haloalkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl or (heterocyclyl)(cycloalkyl)alkyl.

In a preferred embodiment, R² is hydrogen and R¹ is heteroalkyl or vice versa.

In yet a further embodiment, the p38 inhibitor for use in a pharmaceutical combination according to the invention is a compound of formula I wherein R¹ is hydrogen, alkyl, haloalkyl, heteroalkyl or cyanoalkyl.

In yet a further embodiment, the p38 inhibitor for use in a pharmaceutical combination according to the invention is a compound of formula I wherein R¹ is cycloalkyl, cycloalkylalkyl, heteroalkylsubstituted cycloalkyl, heterosubstituted cycloalkyl, heterocyclyl, heterocyclylalkyl or (heterocyclyl)(cycloalkyl)alkyl.

In a preferred embodiment, each of R¹ and R² is independently selected from hydrogen, and hydroxyalkyl, preferably from hydrogen, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl, 2-(hydroxymethyl)-3-hydroxypropyl, 3-hydroxy-1-(2-hydroxyethyl)-propyl and 2-hydroxy-1-methylethyl, more preferably from hydrogen, 2-hydroxyethyl, 2,3-dihydroxypropyl and 1-(hydroxymethyl)2-hydroxyethyl, most preferably from hydrogen, 2-hydroxy-propyl, 3-hydroxy-1-(2-hydroxyethyl)-propyl and 2-hydroxy-1-methylethyl.

In yet a further embodiment, the p38 inhibitor for use in a pharmaceutical combination according to the invention is a compound of formula I wherein R³ is hydrogen, alkyl, cycloalkyl, cycloalkylalkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino) or acyl.

In yet a further embodiment, the p38 inhibitor for use in a pharmaceutical combination according to the invention is a compound of formula I wherein R³ is hydrogen, alkyl, haloalkyl, heteroalkyl, cyanoalkyl, cycloalkyl or cycloalkylalkyl.

In yet a further embodiment, the p38 inhibitor for use in a pharmaceutical combination according to the invention is a compound of formula I wherein R³ is hydrogen, alkyl, haloalkyl, heteroalkyl or cyanoalkyl.

In yet a further embodiment, the p38 inhibitor for use in a pharmaceutical combination according to the invention is a compound of formula I wherein R³ is cycloalkyl or cycloalkylalkyl.

In yet a further embodiment, the p38 inhibitor for use in a pharmaceutical combination according to the invention is a compound of formula I wherein X¹ and X² are both O.

In yet a further embodiment, the p38 inhibitor for use in a pharmaceutical combination according to the invention is a compound of formula I wherein R¹ is alkyl or heteroalkyl.

In yet a further embodiment, the p38 inhibitor for use in a pharmaceutical combination according to the invention is a compound of formula I wherein R¹ is heteroalkyl, preferably 3-hydroxy-1-(2-hydroxyethyl)-propyl or 2-hydroxy-1-methylethyl.

In yet a further embodiment, the p38 inhibitor for use in a pharmaceutical combination according to the invention is a compound of formula I wherein R³ is alkyl or heteroalkyl.

In yet a further embodiment, the p38 inhibitor for use in a pharmaceutical combination according to the invention is a compound of formula I wherein R³ is alkyl, preferably C1-C5 alkyl, more preferably C1-C4 alkyl, more preferably C1-C3 alkyl. In a particularly preferred embodiment, R³ is ethyl or methyl, preferably methyl.

In yet a further embodiment, the p38 inhibitor for use in a pharmaceutical combination according to the invention is a compound of formula I wherein R³ is heteroalkyl, preferably 2-hydroxy-propyl.

In yet a further embodiment, the p38 inhibitor for use in a pharmaceutical combination according to the invention is a compound of formula I wherein W is NH.

In yet a further embodiment, the p38 inhibitor for use in a pharmaceutical combination according to the invention is a compound of formula I wherein Z is N.

In yet a further embodiment, the p38 inhibitor for use in a pharmaceutical combination according to the invention is a compound of formula I wherein Ar¹ is aryl, preferably phenyl optionally substituted with one, two or three halo substituents, most preferably phenyl substituted with two halo substituents in ortho and para position. In a particularly preferred embodiment, Ar¹ is 2,4-difluorophenyl.

In yet a further embodiment, the p38 inhibitor for use in a pharmaceutical combination according to the invention is a compound of formula I wherein X¹ is NR⁴ and X² is NR⁷ or X¹ and X² are each O, wherein R⁴ and R⁷ are as defined above; and wherein
R¹ is hydrogen, alkyl, haloalkyl, cycloalkyl, cycloalkylalkyl, heteroalkylsubstituted cycloalkyl, heterosubstituted cycloalkyl, heteroalkyl, cyanoalkyl, heterocyclyl, heterocyclylalkyl or (heterocyclyl)(cycloalkyl)alkyl; and wherein
R³ is hydrogen, alkyl, cycloalkyl, cycloalkylalkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino) or acyl; and wherein
W is NR², wherein R² is hydrogen, alkyl, acyl or alkoxycarbonyl; and wherein
Ar¹ is aryl; and wherein
Z is N.

In a preferred embodiment, the p38 inhibitor for use in a pharmaceutical combination according to the invention is a compound of formula I wherein X¹ and X² are each O and wherein Z is N and wherein W is NH and wherein Ar¹ is phenyl optionally substituted by one, two or three halo substituents and wherein R¹ is heteroalkyl and wherein R³ is alkyl or heteroalkyl.

Unless otherwise stated, the following terms have the meanings given below:

“Acyl” means a radical —C(O)R, where R is hydrogen, alkyl, cycloalkyl, cycloalkylalkyl, phenyl or phenylalkyl wherein alkyl, cycloalkyl, cycloalkylalkyl, and phenylalkyl are as defined herein. Representative examples include, but are not limited to formyl, acetyl, cylcohexylcarbonyl, cyclohexylmethylcarbonyl, benzoyl, benzylcarbonyl, and the like.

“Acylamino” means a radical-NR′C(O)R, where R′ is hydrogen or alkyl, and R is hydrogen, alkyl, cycloalkyl, cycloalkylalkyl, phenyl or phenylalkyl wherein alkyl, cycloalkyl, cycloalkylalkyl, and phenylalkyl are as defined herein. Representative examples include, but are not limited to formylamino, acetylamino, cylcohexylcarbonylamino, cyclohexylmethyl-carbonylamino, benzoylamino, benzylcarbonylamino, and the like.

“Alkoxy” means a radical —OR where R is an alkyl as defined herein. Examples are methoxy, ethoxy, propoxy, butoxy and the like.

“Alkoxycarbonyl” means a radical R—O—C(O)—, wherein R is an alkyl as defined herein.

“Alkyl” means a linear saturated monovalent hydrocarbon radical of one to six carbon atoms or a branched saturated monovalent hydrocarbon radical of three to six carbon atoms. Examples include methyl, ethyl, propyl, 2-propyl, n-butyl, iso-butyl, tert-butyl, pentyl, and the like. Preferred are C1-C3 alkyl groups, in particular ethyl and methyl.

“Alkylsulfonyl” means a radical R—S(O)₂—, wherein R is alkyl as defined herein.

“Alkylene” means a linear saturated divalent hydrocarbon radical of one to six carbon atoms or a branched saturated divalent hydrocarbon radical of three to six carbon atoms. Examples are methylene, ethylene, 2,2-dimethylethylene, propylene, 2-methylpropylene, butylen, pentylene, and the like.

“Aryl” means a monovalent monocyclic or bicyclic aromatic hydrocarbon radical which is optionally substituted independently with one or more substituents, preferably one, two or three substituents preferably selected from the group consisting of alkyl, hydroxy, alkoxy, haloalkyl, haloalkoxy, Y—C(O)—R (where Y is absent or an alkylene group and R is hydrogen, alkyl, haloalkyl, haloalkoxy, hydroxy, alkoxy, amino, monoalkylamino or dialkylamino), heteroalkyl, heteroalkyloxy, heteroalkylamino, halo, nitro, cyano, amino, monoalkylamino, dialkylamino, alkylsulfonylamino, heteroalkylsulfonylamino, sulfonamido, methylenedioxy, ethylenedioxy, heterocyclyl or heterocyclylalkyl. Monocyclic aryl groups, optionally substituted as described above, are preferred. More specifically, the term aryl includes, but is not limited to, phenyl optionally substituted independently with one, two or three substituents preferably selected from the group consisting of alkyl, hydroxy, alkoxy, haloalkyl, haloalkoxy, Y—C(O)—R (where Y is absent or an alkylene group and R is hydrogen, alkyl, haloalkyl, haloalkoxy, hydroxy, alkoxy, amino, monoalkylamino or dialkylamino), heteroalkyl, heteroalkyloxy, heteroalkylamino, halo, nitro, cyano, amino, monoalkylamino, dialkylamino, alkylsulfonylamino, heteroalkylsulfonylamino, sulfonamido, methylenedioxy, ethylenedioxy, heterocyclyl and heterocyclylalkyl. Particularly preferred aryl groups are substituted phenyl groups selected from chlorophenyl, methoxyphenyl, 2-fluorophenyl, 2,4-difluorophenyl, 1-naphthyl and 2-naphthyl.

“Arylsulfonyl” means a radical R—S(O)₂—, wherein R is aryl as defined herein.

“Aralkyl” refers to an aryl group as defined herein bonded directly through an alkylene group, e.g. benzyl.

“Aryloxy” means a radical —OR where R is an aryl as defined herein, e. g. phenoxy.

“Aryloxycarbonyl” means a radical R—C(═O)— where R is aryloxy, e.g. phenoxycarbonyl.

“Cycloalkyl” refers to a saturated monovalent cyclic hydrocarbon radical of three to seven ring carbons or more specifically those of the specific compounds listed in the enclosed tables or being described in the examples. It is understand that these radicals can be grouped also in a group covering only such radicals but of the first or the second priority application or of both priority applications e. g., cyclopropyl, cyclobutyl, cyclohexyl, 4-methyl-cyclohexyl, and the like.

“Cycloalkylalkyl” means a radical —R^{a}R^{b} where R^{a} is an alkylene group and R^{b} is cycloalkyl group as defined herein, e. g., cyclohexylmethyl, and the like.

“Substituted cycloalkyl” means a cycloalkyl radical as defined herein with one, two or three (preferably one) ring hydrogen atoms independently replaced by cyano or —Y—C(O)R (where Y is absent or an alkylene group and R is hydrogen, alkyl, haloalkyl, hydroxy, alkoxy, amino, monoalkylamino, dialkylamino, or optionally substituted phenyl) or more specifically those of the specific compounds listed in the enclosed tables or being described in the examples.

“Halo” means fluoro, chloro, bromo, or iodo, preferably fluoro and chloro.

“Haloalkyl” means alkyl substituted with one or more same or different halo atoms, e. g. —CH₂Cl, —CF₃, —CH₂CF₃, —CH₂CCl₃, and the like.

“Heteroalkyl” means an alkyl radical as defined herein wherein one, two or three hydrogen atoms have been replaced with a substituent independently selected from the group consisting of —OR^{a}, —N(O)ₙR^{b}R^{c} (where n is 0 or 1 if R^{b} and R^{c} are both independently alkyl, cycloalkyl or cycloalkylalkyl, and 0 if not) and —S(O)ₙR^{d} (where n is an integer from 0 to 2), with the understanding that the point of attachment of the heteroalkyl radical is through a carbon atom, wherein R^{a} is hydrogen, acyl, alkoxycarbonyl, alkyl, cycloalkyl, or cycloalkylalkyl; R^{b} and R^{c} are independently of each other hydrogen, acyl, alkoxycarbonyl, alkyl, cycloalkyl, cycloalkylalkyl, alkylsulfonyl, aminosulfonyl, mono- or dialkylaminosulfonyl, aminoalkyl, mono- or di-alkylaminoalkyl, hydroxyalkyl, alkoxyalkyl, hydroxyalkylsulfonyl or alkoxyalkylsulfonyl; and when n is 0, R^{d} is hydrogen, alkyl, cycloalkyl, cycloalkylalkyl or optionally substituted phenyl, and when n is 1 or 2, R^{d} is alkyl, cycloalkyl, cycloalkylalkyl, optionally substituted phenyl, amino, acylamino, monoalkylamino, or dialkylamino. Preferred heteroalkyl groups include hydroxyalkyl groups, preferably C1-C6 hydroxyalkyl groups.

Representative examples include, but are not limited to, 2-hydroxyethyl, 2-hydroxy-propyl, 3-hydroxypropyl, 2-hydroxy-1-hydroxymethylethyl, 2-hydroxy-1-methylethyl, 2,3-dihydroxypropyl, 1-hydroxymethylethyl, 3-hydroxybutyl, 2,3-dihydroxybutyl, 2-hydroxy-1-methylpropyl, 3-hydroxy-1-(2-hydroxyethyl)-propyl, 2-aminoethyl, 3-aminopropyl, 2-methylsulfonylethyl, aminosulfonylmethyl, aminosulfonylethyl, aminosulfonylpropyl, methylaminosulfonylmethyl, methylaminosulfonylethyl, methylaminosulfonylpropyl, and the like. Particularly preferred heteroalkyl groups are 2-hydroxy-propyl, 3-hydroxy-1-(2-hydroxyethyl)-propyl or 2-hydroxy-1-methylethyl.

“Hydroxyalkyl” means an alkyl radical as defined herein, substituted with one or more, preferably one, two or three hydroxy groups, provided that the same carbon atom does not carry more than one hydroxy group. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 2-hydroxy-1-hydroxymethylethyl, 2,3-dihydroxybutyl, 3,4-dihydroxybutyl, 2-(hydroxymethyl)-3-hydroxypropyl, 3-hydroxy-1-(2-hydroxyethyl)-propyl and 2-hydroxy-1-methylethyl, preferably 2-hydroxyethyl, 2,3-dihydroxypropyl and 1-(hydroxymethyl)₂-hydroxyethyl, more preferably 2-hydroxy-propyl, 3-hydroxy-1-(2-hydroxyethyl)-propyl and 2-hydroxy-1-methylethyl. Accordingly, as used herein, the term “hydroxyalkyl” is used to define a subset of heteroalkyl groups.

“Heteroalkylcarbonyl” means the group R^{a}—C(═O)—, where R^{a} is a heteroalkyl group. Representative examples include acetyloxymethylcarbonyl, aminomethylcarbonyl, 4-acetyloxy-2,2-dimethyl-butan-2-oyl, 2-amino-4-methyl-pentan-2-oyl, and the like.

“Heteroalkyloxy” means the group R^{a}—O—, where R^{a} is a heteroalkyl group. Representative examples include (Me-C(═O)—O—CH₂—O—, and the like.

“Heteroalkyloxycarbonyl” means the group R^{a}—C(═O), where R^{a} is heteroalkyloxy. Representative examples include 1-acetyloxy-methoxycarbonyl (Me-C(═O)—OCH₂—O—C(═O)—) and the like.

“Heteroaryl” means a monovalent monocyclic or bicyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring is optionally substituted independently with one or more substituents, preferably one or two substituents, selected from alkyl, haloalkyl, heteroalkyl, hydroxy, alkoxy, halo, nitro or cyano. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl or benzothienyl, imidazo[1,2-a]-pyridinyl, imidazo[2,1-b]thiazolyl, and the derivatives thereof.

“Heteroaralkyl” means a radical —R^{a}R^{b} where R^{a} is an alkylene group and R^{b} is a heteroaryl group, e. g. pyridin-3-ylmethyl, imidazolylethyl, pyridinylethyl, 3-(benzofuran-2-yl)propyl, and the like.

“Heteroalkylsubstituted cycloalkyl” means a cycloalkyl radical as defined herein wherein one, two or three hydrogen atoms in the cycloalkyl radical have been replaced with a heteroalkyl group with the understanding that the heteroalkyl radical is attached to the cycloalkyl radical via a carbon-carbon bond. Representative examples include, but are not limited to, 1-hydroxymethylcyclopentyl, 2-hydroxymethylcyclohexyl, and the like.

“Heterosubstituted cycloalkyl” means a cycloalkyl radical as defined herein wherein one, two or three hydrogen atoms in the cycloalkyl radical have been replaced with a substituent independently selected from the group consisting of hydroxy, alkoxy, amino, acylamino, monoalkylamino, dialkylamino, oxo(C═O), imino, hydroximino (═NOH), NR′SO₂R^{d} (where R′ is hydrogen or alkyl and R^{d} is alkyl, cycloalkyl, hydroxyalkyl, amino, monoalkylamino or dialkylamino), —X—Y—C(O)R (where X is O or NR′, Y is alkylene or absent, R is hydrogen, alkyl, haloalkyl, alkoxy, amino, monoalkylamino, dialkylamino, or optionally substituted phenyl, and R′ is H or alkyl), or —S(O)ₙR (where n is an integer from 0 to 2) such that when n is 0, R is hydrogen, alkyl, cycloalkyl, cycloalkylalkyl optionally substituted phenyl or thienyl, and when n is 1 or 2, R is alkyl, cycloalkyl, cycloalkylalkyl, optionally substituted phenyl, thienyl, amino, acylamino, monoalkylamino or dialkylamino. Representative examples include, but are not limited to, 2-, 3-, or 4-hydroxycyclohexyl, 2-, 3-, or 4-aminocyclohexyl, 2-, 3-, or 4-methanesulfonamido-cyclohexyl, and the like, preferably 4-hydroxycyclohexyl, 2-aminocyclohexyl or 4-methanesulfonamido-cyclohexyl.

“Heterosubstituted cycloalkyl-alkyl” means a radical R^{a}R^{b}—where R^{a} is a heterosubstituted cycloalkyl radical and R^{b} is an alkylene radical.

“Heterocycloamino” means a saturated monovalent cyclic group of 4 to 8 ring atoms, wherein one ring atom is N and the remaining ring atoms are C. Representative examples include piperidine and pyrrolidine.

“Heterocyclyl” means a saturated or unsaturated non-aromatic cyclic radical of 3 to 8 ring atoms in which one or two ring atoms are heteroatoms selected from N, O, or S(O)ₙ (where n is an integer from 0 to 2), the remaining ring atoms being C, where one or two C atoms may optionally be replaced by a carbonyl group. The heterocyclyl ring may be optionally substituted independently with one, two, or three substituents selected from alkyl, haloalkyl, heteroalkyl, halo, nitro, cyano, cyanoalkyl, hydroxy, alkoxy, amino, monoalkylamino, dialkylamino, aralkyl, —(X)ₙ—C(O)R (where X is O or NR′, n is 0 or 1, R is hydrogen, alkyl, haloalkyl, hydroxy (when n is 0), alkoxy, amino, monoalkylamino, dialkylamino, or optionally substituted phenyl, and R′ is H or alkyl), -alkylene-C(O)R^{a} (where R^{a} is alkyl, OR or NR′R″ and R is hydrogen, alkyl or haloalkyl, and R′ and R″ are independently hydrogen or alkyl), or —S(O)ₙR (where n is an integer from 0 to 2) such that when n is 0, R is hydrogen, alkyl, cycloalkyl, or cycloalkylalkyl, and when n is 1 or 2, R is alkyl, cycloalkyl, cycloalkylalkyl, amino, acylamino, monoalkylamino, dialkylamino or heteroalkyl. More specifically the term heterocyclyl includes, but is not limited to, tetrahydropyranyl, piperidino, N-methylpiperidin-3-yl, piperazino, N-methylpyrrolidin-3-yl, 3-pyrrolidino, morpholino, thiomorpholino, thiomorpholino-1-oxide, thiomorpholino-1,1-dioxide, 4-(1,1-dioxo-tetrahydro-2H-thiopyranyl), pyrrolinyl, imidazolinyl, N-methanesulfonyl-piperidin-4-yl, and the derivatives thereof.

“Heterocyclylalkyl” means a radical —R^{a}R^{b} where R^{a} is an alkylene group and R^{b} is a heterocyclyl group as defined above, e. g. tetrahydropyran-2-ylmethyl, 2- or 3-piperidinylmethyl, 3-(4-methyl-piperazin-1-yl)propyl and the like.

“(Heterocyclyl)(cycloalkyl)alkyl” means an alkyl radical wherein two hydrogen atoms have been replaced with a heterocyclyl group and a cycloalkyl group.

“(Heterocyclyl)(heteroaryl)alkyl” means an alkyl radical wherein two hydrogen atoms have been replaced with a heterocycyl group and a heteroaryl group.

“Amino” means a radical —NH₂.

“Monoalkylamino” means a radical —NHR where R is an alkyl, hydroxyalkyl, cycloalkyl, or cycloalkylalkyl group as defined above, e. g. methylamino, (1-methylethyl) amino, hydroxymethylamino, cyclohexylamino, cyclohexylmethylamino, cyclohexylethylamino, and the like.

“Dialkylamino” means a radical —NRR′ where R and R′ independently represent an alkyl, hydroxyalkyl, cycloalkyl, or cycloalkylalkyl group as defined herein. Representative examples include, but are not limited to dimethylamino, methylethylamino, di(1-methylethyl)amino, (methyl)(hydroxymethyl)amino, (cyclohexyl)(methyl)amino, (cyclohexyl)(ethyl)amino, (cyclohexyl)(propyl)amino, (cyclohexylmethyl)(methyl)amino, (cyclohexylmethyl)(ethyl)amino, and the like.

“Optionally substituted phenyl” means a phenyl ring which is optionally substituted independently with one or more substituents, preferably one, two or three substituents, more preferably two substituents selected from the group consisting of alkyl, hydroxy, alkoxy, haloalkyl, haloalkoxy, heteroalkyl, halo, nitro, cyano, amino, methylenedioxy, ethylenedioxy, and acyl, preferably halo, most preferably fluoro.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxy-benzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-enel-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e. g. an alkaline metal ion, an alkaline earth metal ion, or an aluminum ion; or coordinates with an organic base such as ethanolamine, diethanolamine, triethanolamine, tromethamine, N-methylglucamine, and the like. Preferred salts comprise acid addition salts formed with hydrochloric acid.

### Hearing Loss and Related Conditions

The term “hearing loss” which is used herein interchangeably with the term “hearing impairment” refers to a diminished sensitivity to the sounds normally heard by a subject. The severity of a hearing loss is categorized according to the increase in volume above the usual level necessary before the listener can detect it. The term “hearing loss” as used herein includes sudden hearing loss (SHL) which is indicated in the literature also as “sudden sensorineural hearing loss (SSHL)”. SHL refers to illness which is characterized by a sudden, rapid sensorineural hearing loss mostly in one ear without obvious causes, normally accompanied with dizziness, and without vestibular symptomatology. SHL is defined as greater than 30 dB hearing reduction, over at least three contiguous frequencies, occurring over a period of 72 hours or less. SHL can be caused e.g. by unspecific stress.

Hearing loss as referred herein is defined as a diminished ability to hear sounds like other people do. This can be caused either by conductive hearing loss, sensorineural hearing loss or a combination of both.

Conductive hearing loss means that the vibrations are not passing through from the outer ear to the inner ear, specifically the cochlea. It can be due to an excessive build-up of earwax, glue ear, an ear infection with inflammation and fluid buildup, a perforated or defective eardrum, or a malfunction of the ossicles (bones in the middle ear).

Sensorineural hearing loss is caused by dysfunction of the inner ear, the cochlea, auditory nerve, or brain damage. Usually, this kind of hearing loss is due to damage of the hair cells in the cochlea.

Hearing loss as referred herein is usually sensorineural hearing loss or a combination of conductive hearing loss and sensorineural hearing loss. Sensorineural hearing loss can be related to age, to an acute or constant exposure to noise or chemicals, to a brain trauma or non specific stress which may lead to sudden hearing loss.

The term “hair cell degeneration” as used herein refers to a gradual loss of hair cell function and integrity and/or leading ultimately to hair cell death.

The term “hair cell death” as used herein refers to apoptosis of the hair cells in the inner ear.

The terms “identification of hair cell damage” or “detection of hair cell damage” which are used interchangeably herein refer to a method by which the degree of hair cell damage in the inner ear can be determined. Such methods are known in the art and comprise for example fluorescent imaging of the hair cells, as described in the examples. An audiogram that demonstrates loss of hearing sensitivity at moderate to high frequencies is also indicative of hair cell damage. A decrease of hearing potential with no subsequent recovery is also diagnostic of hair cell damage.

The term “chemically induced hearing loss” or “hearing loss induced by a chemical” as referred herein refers to hearing loss which is induced and/or caused by chemical agents such as solvents, gases, paints, heavy metals, and/or medicaments which are ototoxic.

The term “sound pressure level (SPL)” or “acoustic pressure level” as referred herein is a logarithmic measure of the effective sound pressure of a sound relative to a reference value. Sound pressure level, denoted Lₚ and measured in dB, above a standard reference level, is given by:

Lₚ=10 log₁₀(pᵣₘₛ²/p₀²)=20 log₁₀(pᵣₘₛ/p₀)dB(SPL)

where pᵣₘₛ is the root mean square sound pressure, measured in Pa and p₀ is the reference sound pressure, measured in Pa. The commonly used reference sound pressure in air is p₀=20 μPa (Root Mean Squared) or 0.0002 dynes/cm², which is usually considered the threshold of human hearing.

In one aspect, the present invention provides a pharmaceutical combination comprising a PPAR agonist and a compound of formula I or II for use in a method of preventing or treating hearing loss in a subject. In a further aspect of the invention, the present invention provides a method of preventing or treating hearing loss in a subject, which method comprises administering to the subject pharmaceutical combination comprising a PPAR agonist and a compound of formula I or II. In a further aspect, the present invention provides a pharmaceutical combination comprising a PPAR agonist and a p38 inhibitor for use in a method of preventing or treating hearing loss in a subject. In a further aspect of the invention, the present invention provides a method of preventing or treating hearing loss in a subject, which method comprises administering to the subject pharmaceutical combination comprising a PPAR agonist and a p38 inhibitor. In some embodiments, the pharmaceutical combination is administered to the subject in an amount that is sufficient to prevent or treat hearing loss in the subject. In a further aspect, the present invention provides the use of a pharmaceutical combination comprising a PPAR agonist and a compound of formula I or II for the manufacture of a medicament for preventing or treating hearing loss in a subject. In a further aspect, the present invention provides the use of a pharmaceutical combination comprising a PPAR agonist and a p38 inhibitor for the manufacture of a medicament for preventing or treating hearing loss in a subject. In a further aspect, the present invention provides the use of a pharmaceutical combination comprising a PPAR agonist and a compound of formula I or II for preventing or treating hearing loss in a subject. In a further aspect, the present invention provides the use of a pharmaceutical combination comprising a PPAR agonist and a p38 inhibitor for preventing or treating hearing loss in a subject.

In some preferred embodiments, hearing loss to be prevented or treated by the methods of the present invention is caused by a noise trauma, by a medical intervention, by ischemic injury, by age or is chemically induced. The hearing loss can be thus a consequence of a medical intervention such as e.g. cochlear implantation. The chemical induction is usually caused by a chemical agent e.g. by an antibiotic or a chemotherapeutic agent. In some preferred embodiments hearing loss is sudden hearing loss. Hearing loss caused by age comprises e.g. presbycusis. Preferably hearing loss caused by a noise trauma, cochlear implantation, or which is chemically induced, preferably by an antibiotic, is prevented or treated by the methods of the present invention. More preferably, hearing loss caused by a noise trauma or which is chemically induced, preferably by an antibiotic, is prevented or treated by the methods of the present invention. In some embodiments, hearing loss is of sensorineural origin caused by a damage leading to malnutrition of the cells in early brain development. In this case early treatment with a PPAR agonist can be disease modifying preventing further damage.

In some embodiments, the pharmaceutical combination of the invention is administered before the subject has developed or before it is at risk to develop hearing loss, hair cell degeneration, hair cell death and/or a condition characterized by hair cell damage. In some embodiments, the pharmaceutical combination of the invention is administered after the subject has acquired hearing loss, hair cell degeneration, hair cell death and/or a condition characterized by hair cell damage.

Further diseases, disorders or conditions which are related to, caused or characterized by hair cell degeneration and/or hair cell death and which can be prevented or treated by the methods of the present invention are e.g. Menibre's disease, acute peripheral vestibulopathy and tinnitus.

Thus in some embodiments the present invention provides a pharmaceutical combination described herein for use in a method of preventing or inhibiting hair cell degeneration or hair cell death in a subject, wherein hair cell degeneration or hair cell death is related to and/or caused by Menière's disease, acute peripheral vestibulopathy and/or tinnitus.

In some embodiments the present invention provides a pharmaceutical combination described herein for use in a method of preventing or treating Menière's disease in a subject.

In some embodiments, the present invention provides a method of preventing or treating Menière's disease in a subject which method comprises administering to the subject a pharmaceutical combination described herein.

In some embodiments, the present invention provides the use of a pharmaceutical combination described herein for the manufacture of a medicament for preventing or treating Menière's disease in a subject.

In some embodiments, the present invention provides the use of a pharmaceutical combination described herein for preventing or treating Menibre's disease in a subject.

In some embodiments the present invention provides a pharmaceutical combination described herein for use in a method of preventing or treating acute peripheral vestibulopathy in a subject.

In some embodiments, the present invention provides a method of preventing or treating acute peripheral vestibulopthy in a subject which method comprises administering to the subject a pharmaceutical combination described herein.

In some embodiments, the present invention provides the use of a pharmaceutical combination described herein for the manufacture of a medicament for preventing or treating acute peripheral vestibulopthy in a subject.

In some embodiments, the present invention provides the use of a pharmaceutical combination described herein for preventing or treating acute peripheral vestibulopthy in a subject.

In some embodiments the present invention provides a pharmaceutical combination described herein for use in a method of preventing or treating tinnitus in a subject.

In some embodiments, the present invention provides a method of preventing or treating tinnitus in a subject which method comprises administering to the subject a pharmaceutical combination described herein.

In some embodiments, the present invention provides the use of a pharmaceutical combination described herein for the manufacture of a medicament for preventing or treating tinnitus in a subject.

In some embodiments, the present invention provides the use of a pharmaceutical combination described herein for preventing or treating tinnitus in a subject.

### Hearing Loss, Hair Cell Degeneration or Hair Cell Death Caused by a Noise Trauma or by Medical Intervention

Exposure to loud noise causes noise-induced hearing loss (NIHL) by damaging the organs of Corti. Damage by NIHL depends upon both the level of the noise and the duration of the exposure. Hearing loss may be temporary (temporary threshold shift, TTS) if a repair mechanism is able to restore the organ of the Corti. However, it becomes permanent (permanent threshold shift, PTS) when hair cells or neurons die. Structural modifications correlated to noise trauma are of two types: (1) mild damage of synapses and or hair cell stereocilia which can be repaired by cellular repair mechanisms and accounts for TTS and recovery and (2) severe damage inducing hair cell and neuronal apoptosis which can not be repaired by cellular repair mechanisms and accounts for PTS.

A noise trauma as referred herein is a noise trauma which is sufficient to cause damage to the organs of corti, in particular a noise trauma causing temporary or permanent hearing loss. A noise trauma can be caused by exposure to a sound pressure level of e.g., at least 70 dB (SPL), at least 90 dB (SPL), at least 100 dB (SPL), at least 120 dB (SPL) or at least 130 dB (SPL). Hearing loss can also be caused by a medical intervention usually by a medical intervention in the ear e.g. by cochlea surgery such as cochlear implantation.

In some embodiments the pharmaceutical combination of the invention is administered before the subject is exposed to a noise trauma or medical intervention. In some embodiments, the pharmaceutical combination of the invention is administered after the subject is exposed to a noise trauma or medical intervention. In a particular embodiment the pharmaceutical combination of the invention is administered prior to cochlear surgery i.e. before the subject undergoes cochlear surgery.

### Hearing Loss, Hair Cell Degeneration or Hair Cell Death Caused by Age

Hearing loss caused by age also referred in the literature as “age-related hearing loss” is the cumulative effect of aging on hearing. It is normally a progressive bilateral symmetrical age-related sensorineural hearing loss. The hearing loss is most marked at higher frequencies.

There are four pathological types of hearing loss caused by age:

1) sensory: characterised by degeneration of organs of corti. 2) neural: characterised by degeneration of cells of spiral ganglion. 3) strial/metabolic: characterised by atrophy of stria vascularis in all turns of cochlea. 4) cochlear conductive: due to stiffening of the basilar membrane thus affecting its movement.

Hearing loss caused by age to be prevented or treated by the methods of the present invention is usually related to the first pathological type i.e. hearing loss characterised by degeneration of the organ of Corti. Thus, in some embodiments the pharmaceutical combination of the invention is administered to the subject prior to degeneration of the organ of Corti, e.g. prior to damage or apoptosis of hair cells and/or prior to hair cell degeneration or hair cell death.

### Chemically Induced Hearing Loss, Hair Cell Degeneration or Hair Cell Death

Hearing loss, hair cell degeneration or hair cell death can be induced chemically i.e. by a chemical agent e.g. by an antibiotic, a drug, a chemotherapeutic agent, heavy metals or organic agents. Antibiotics which may cause hearing loss include for example cephalosporins such as cephalexin (Keflex), cefaclor (Ceclor), and cefixime (Suprax); aminoglycosides such as gentamycin, tobramycin and streptomycin; macrolides, such as erythromycin, azithromycin (Zithromax) and clarithromycin; sulfonamides such as trimethoprim-sulfamethoxazole or tetracylines such as tetracycline, or doxycycline. In particular hearing loss, hair cell degeneration or hair cell death is effectively prevented or treated by the methods of the present invention in a subject exposed to gentamycin.

Chemotherapeutic agents, e.g. anti-cancer agents which may cause hearing loss, hair cell degeneration or hair cell death include for example platinum-containing agents e.g. cisplatin, and carboplatin, preferably cisplatin. Drugs which may cause hearing loss, hair cell degeneration or hair cell death include for example furosemide, quinine, aspirin and other salicylates. Heavy metals which may cause hearing loss include for example mercury, lead. Organic agents which may cause hearing loss, hair cell degeneration or hair cell death include for example toluene, xylene, or styrene. In some embodiments, the pharmaceutical combination of the invention is administered to the subject before the subject is exposed to a chemical agent, thereby preventing the subject from chemically induced hearing loss, hair cell degeneration or hair cell death. In some embodiments, the pharmaceutical combination of the invention is administered to the subject after the subject is exposed to a chemical agent thereby treating the subject having chemically induced hearing loss, hair cell degeneration or hair cell death.

In a preferred embodiment, when hearing loss is caused by a noise trauma or is chemically induced, the pharmaceutical combination of the invention is administered to the subject prior to exposure of the subject to the noise trauma or to the chemical wherein at least 50%, preferably at least 60%, more preferably at least 70%, most preferably at least 80%, in particular at least 90%, more particular at least 95%, even more particular at least 99%, most particular 100% of the cell damage of the hair cells caused by the noise trauma or the chemical agent is prevented.

In one aspect, the present invention provides a pharmaceutical combination comprising PPAR agonist and a compound of formula I or II for use in a method of preventing or inhibiting hair cell degeneration or hair cell death in a subject. In a further aspect, the present invention provides a pharmaceutical combination comprising PPAR agonist and a a p38 inhibitor for use in a method of preventing or inhibiting hair cell degeneration or hair cell death in a subject. In a further aspect, the present invention provides a method of preventing or inhibiting hair cell degeneration or hair cell death in a subject, which method comprises administering to the subject a pharmaceutical combination described herein. In some embodiments the pharmaceutical combination of the invention is administered to the subject in an amount that is sufficient to prevent or inhibit hair cell degeneration or hair cell death in the subject. In a further aspect, the present invention provides the use of a pharmaceutical combination described herein for the manufacture of a medicament for preventing or inhibiting hair cell degeneration or hair cell death in a subject. In a further aspect, the present invention provides the use of a pharmaceutical combination described herein for preventing or inhibiting hair cell degeneration or hair cell death in a subject. In some embodiments, hair cell degeneration or hair cell death in a subject is caused by a noise trauma, by age, a medical intervention, sudden hearing loss, or ischemic events such as ischemic injury, or is chemically induced wherein the chemical induction is caused by e.g. an antibiotic or a chemotherapeutic agent. Noise trauma, age, a medical intervention, sudden hearing loss, or ischemic events, or chemical induction can cause hair cell degeneration or hair cell death in a subject as described above for methods of preventing or treating hearing loss.

In some embodiments hearing loss, hair cell degeneration or hair cell death is caused by hair cell damage. In some embodiments the pharmaceutical combination of the invention is administered to the subject prior to identification of said hair cell damage i.e. prior to occurrence of hair cell damage. In a preferred embodiment when hair cell damage is caused by a noise trauma or is chemically induced, the pharmaceutical combination of the invention is administered to the subject prior to exposure of the subject to the noise trauma or to the chemical agent wherein at least 50%, preferably at least 60%, more preferably at least 70%, in particular at least 80%, more particular at least 90% of the cell damage of the hair cells caused by the noise trauma or the chemical agent is prevented. Identification/occurrence of hair cell damage is usually determined by evaluation of the state of the hair cells which can be easily accomplished as described above or as disclosed in the examples.

### Pharmaceutical Combinations

As outlined above, in one aspect, the present invention provides a pharmaceutical combination comprising:

(a) a PPAR agonist;
(b) a p38 kinase inhibitor; and optionally
(c) one or more pharmaceutically acceptable diluents, excipients or carriers
for use in a method of preventing or treating hearing loss in a subject and/or for use in a method of preventing or inhibiting hair cell degeneration or hair cell death in a subject.

The term “hearing loss”, “hair cell degeneration” and “hair cell death” are as defined above.

Useful PPAR agonists are as defined above. In a preferred embodiment, said PPAR agonist is a PPAR gamma agonist, preferably a micronized PPAR gamma agonist, in particular pioglitazone or a pharmaceutically acceptable salt thereof. In a particularly preferred embodiment, said PPAR agonist is pioglitazone hydrochloride, preferably micronized pioglitazone hydrochloride.

Useful p38 kinase inhibitors are as defined above. In a preferred embodiment, said p38 kinase inhibitors are inhibiting p38-alpha, p38-beta, p38-gamma or p38-delta or combinations thereof, preferably inhibiting p38-alpha and/or p38-beta, more preferably inhibiting p38-alpha. Further useful p38 kinase inhibitors are compounds of the formula I or II as defined supra. Further useful p38 kinase inhibitors are p38 kinase inhibitors selected from the group consisting of pamapimod, losmapimod, dilmapimod, AZD7624, ARRY-371797, LY2228820, R9111, PH-797804, BIRB 796, VX-702, VX-745, SB 239063, SB202190, SCIO 469, and BMS 582949, preferably p38 kinase inhibitors selected from the group consisting of pamapimod, losmapimod, dilmapimod, ARRY-371797, LY2228820, R9111, PH-797804, BIRB 796, VX-702, VX-745, SB 239063, SB202190, SCIO 469, and BMS 582949, in particular pamapimod and/or R9111, more particular pamapimod or a pharmaceutically acceptable salt thereof.

A pharmaceutical combination according to the invention is for example a combined preparation or a pharmaceutical composition, for simultaneous, separate or sequential use. The term “combined preparation” as used herein defines especially a “kit of parts” in the sense that said PPAR agonist and said p38 inhibitor can be dosed independently, either in separate form or by use of different fixed combinations with distinguished amounts of the active ingredients. The ratio of the amount of PPAR agonist to the amount of p38 inhibitor to be administered in the combined preparation can be varied, e.g. in order to cope with the needs of a patient sub-population to be treated or the needs of a single patient, which needs can be different due to age, sex, body weight, etc. of a patient. The individual parts of the combined preparation (kit of parts) can be administered simultaneously or sequentially, i.e. chronologically staggered, e.g. at different time points and with equal or different time intervals for any part of the kit of parts.

The term “pharmaceutical composition” refers to a fixed-dose combination (FDC) that includes the PPAR agonist and the p38 inhibitor combined in a single dosage form, having a predetermined combination of respective dosages.

The pharmaceutical combination further may be used as add-on therapy. As used herein, “add-on” or “add-on therapy” means an assemblage of reagents for use in therapy, the subject receiving the therapy begins a first treatment regimen of one or more reagents prior to beginning a second treatment regimen of one or more different reagents in addition to the first treatment regimen, so that not all of the reagents used in the therapy are started at the same time. For example, adding p38 inhibitor therapy to a patient already receiving PPAR agonist therapy and vice versa.

In a particularly preferred embodiment, the pharmaceutical combination according to the invention is a pharmaceutical composition, i.e. a fixed-dose combination.

In a further preferred embodiment, the pharmaceutical combination according to the invention is a combined preparation.

The amount of the PPAR agonist and the p38 inhibitor to be administered will vary depending upon factors such as the particular compound, disease condition and its severity, according to the particular circumstances surrounding the case, including, e.g., the specific PPAR agonist being administered, the route of administration, the condition being treated, the target area being treated, and the subject or host being treated.

In one embodiment, the invention provides a pharmaceutical combination comprising a PPAR agonist and a p38 inhibitor, wherein said PPAR agonist and said p38 inhibitor are present in a therapeutically effective amount.

In another preferred embodiment, the invention provides a pharmaceutical combination comprising a PPAR agonist and a p38 inhibitor, wherein said PPAR agonist and said p38 inhibitor are present in an amount producing an additive therapeutic effect.

As used herein, the term “additive” means that the effect achieved with the pharmaceutical combinations of this invention is approximately the sum of the effects that result from using the individual agents, namely the PPAR agonist and the p38 inhibitor, as a monotherapy. Advantageously, an additive effect provides for greater efficacy at the same doses, and may lead to longer duration of response to the therapy.

In a preferred embodiment, the invention provides a pharmaceutical combination comprising a PPAR agonist and a p38 inhibitor, wherein said PPAR agonist and said p38 inhibitor produce a synergistic therapeutic effect i.e. wherein said PPAR agonist and said p38 inhibitor are present in an amount producing a synergistic therapeutic effect.

As used herein, the term “synergistic” means that the effect achieved with the pharmaceutical combinations of this invention is greater than the sum of the effects that result from using the agents, namely the PPAR agonist and the p38 inhibitor, as a monotherapy. Advantageously, such synergy provides greater efficacy at the same doses.

In one embodiment, the invention provides a pharmaceutical combination comprising a p38 inhibitor and a PPAR agonist, wherein the amount of said PPAR agonist in the combination is from about 0.1 to about 50 mg or from about 0.1 to about 45 mg or from about 0.1 to about 30 mg or from about 0.1 to about 15 mg or from about 0.1 to about 12 mg or from about 0.1 to about 8 mg or from about 6 to about 12 mg. In a preferred embodiment, the amount of said PPAR agonist in the combination is from about 5 to about 15 mg, preferably about 12 mg.

In a preferred embodiment, the invention provides a pharmaceutical combination comprising a p38 inhibitor and a PPAR agonist, wherein the amount of said PPAR agonist in the combination is about 1 mg, about 2 mg, about 3 mg, about 4 mg, about 5 mg, about 6 mg, about 7 mg, about 8 mg, about 9 mg, about 10 mg, about 11 mg or about 12 mg, preferably about 12 mg.

In another preferred embodiment, the invention provides a pharmaceutical combination comprising a p38 inhibitor and a PPAR agonist, wherein the amount of said PPAR agonist in the combination is from about 0.1 to about 10 mg, preferably from about 0.1 to about 5 mg, more preferably from about 0.1 to about 1.5 mg, in particular from about 0.1 to about 1 mg.

In a preferred embodiment, the invention provides a pharmaceutical combination comprising a p38 inhibitor and a PPAR agonist, wherein the amount of said PPAR agonist in the combination is from about 0.05 μM to about 20 μM, preferably from about 0.05 μM to about μM, more preferably from about 0.05 μM to about 4 μM, in particular from about 0.05 μM to about 2 μM.

In a particularly preferred embodiment, the invention provides a pharmaceutical combination comprising a p38 inhibitor, preferably a micronized p38 inhibitor and pioglitazone or a pharmaceutically acceptable salt thereof, preferably pioglitazone hydrochloride, even more preferably micronized pioglitazone hydrochloride, wherein the amount of pioglitazone in the combination is about 1 mg, about 2 mg, about 3 mg, about 4 mg, about 5 mg, about 6 mg, about 7 mg, about 8 mg, about 9 mg, about 10 mg, about 11 mg or about 12 mg, preferably about 12 mg.

In another particularly preferred embodiment, the invention provides a pharmaceutical combination comprising a p38 inhibitor, preferably a micronized p38 inhibitor and pioglitazone or a pharmaceutically acceptable salt thereof, preferably pioglitazone hydrochloride, even more preferably micronized pioglitazone hydrochloride, wherein the amount of micronized pioglitazone hydrochloride in the combination is from about 0.1 to about 10 mg, preferably from about 0.1 to about 5 mg, more preferably from about 0.1 to about 1.5 mg, in particular from about 0.1 to about 1 mg.

In another particularly preferred embodiment, the invention provides a pharmaceutical combination comprising a p38 inhibitor, preferably a micronized p38 inhibitor and pioglitazone or a pharmaceutically acceptable salt thereof, preferably pioglitazone hydrochloride, even more preferably micronized pioglitazone hydrochloride, wherein the amount of micronized pioglitazone hydrochloride in the combination is from about 0.05 μM to about 20 μM, preferably from about 0.05 μM to about 10 μM, more preferably from about 0.05 μM to about 4 μM, in particular from about 0.05 μM to about 2 μM.

In a further particularly preferred embodiment, the invention provides a pharmaceutical combination comprising a p38 inhibitor, preferably a micronized p38 inhibitor and micronized pioglitazone hydrochloride, wherein the amount of micronized pioglitazone hydrochloride in the combination is about 12 mg (e.g. about 12.2 mg).

In a preferred embodiment, the invention provides a pharmaceutical combination comprising a p38 inhibitor and pioglitazone or a pharmaceutically acceptable salt thereof, wherein the amount of pioglitazone or a pharmaceutically acceptable salt thereof in the combination is below the dose typically needed for the treatment of diabetes with pioglitazone or a pharmaceutically acceptable salt thereof (vide infra).

In one embodiment, the invention provides a pharmaceutical combination comprising a p38 inhibitor and a PPAR agonist, wherein the amount of said p38 inhibitor in the combination is from about 1 to about 500 mg or from about 1 to about 450 mg or from about 1 to about 400 mg or from about 1 to about 350 mg or from about 1 to about 300 mg or from about 1 to about 250 mg or from about 1 to about 200 mg or from about 1 to about 150 mg or from about 1 to about 125 mg or from about 10 to about 125 mg or from about 10 to about 100 mg or from about 20 to about 100 mg or from about 30 to about 100 mg or from about 40 to about 100 mg or from about 50 to about 100 mg, or from about 1 to about 100 mg or from about 2 to about 50 mg or from about 6 to about 30 mg.

In one embodiment, the invention provides a pharmaceutical combination comprising a p38 inhibitor and a PPAR agonist, wherein the amount of said p38 inhibitor in the combination is from about 0.1 to about 50 mg, preferably from about 0.1 to about 20 mg, more preferably from about 0.1 to about 10 mg, in particular from about 0.1 to about 5 mg, more particular from about 0.1 to about 1 mg.

In a preferred embodiment, the invention provides a pharmaceutical combination comprising a p38 inhibitor and a PPAR agonist, wherein the amount of said p38 inhibitor in the combination is from about 0.05 μM to about 50 μM, preferably from about 0.05 μM to about μM, more preferably from about 0.05 μM to about 10 μM, in particular from about 0.05 μM to about 5 μM, more particular from about 0.05 μM to about 2 μM.

In a preferred embodiment, the invention provides a pharmaceutical combination comprising a p38 inhibitor and a PPAR agonist, wherein the amount of said p38 inhibitor in the combination is about 25 mg, about 50 mg, about 75 mg, about 125 mg, about 150 mg or about 300 mg.

In a further preferred embodiment, the invention provides a pharmaceutical combination comprising a p38 inhibitor and a PPAR agonist, wherein the amount of said p38 inhibitor in the combination is about 2 mg, about 6 mg, about 12 mg, about 25 mg, about 50 mg or about 75 mg.

In one embodiment, the invention provides a pharmaceutical combination comprising a p38 inhibitor and a PPAR agonist, wherein the amount of said PPAR agonist in the combination is from 0.1 to about 50 mg or from about 0.1 to about 45 mg or from about 0.1 to about 30 mg or from about 0.1 to about 15 mg or from about 0.1 to about 12 mg or from about 0.1 to about 8 mg or from about 6 to about 12 mg; and wherein the amount of said p38 inhibitor in the combination is from about 1 to about 500 mg or from about 1 to about 450 mg or from about 1 to about 400 mg or from about 1 to about 350 mg or from about 1 to about 300 mg or from about 1 to about 250 mg or from about 1 to about 200 mg or from about 1 to about 150 mg or from about 1 to about 125 mg or from about 10 to about 125 mg or from about 10 to about 100 mg or from about 20 to about 100 mg or from about 30 to about 100 mg or from about 40 to about 100 mg or from about 50 to about 100 mg.

In a preferred embodiment, the invention provides a pharmaceutical combination comprising a p38 inhibitor and a PPAR agonist, wherein the amount of said PPAR agonist in the combination is about 1 mg, about 2 mg, about 3 mg, about 4 mg, about 5 mg, about 6 mg, about 7 mg, about 8 mg, about 9 mg, about 10 mg, about 11 mg or about 12 mg; and wherein the amount of said p38 inhibitor in the combination is about 2 mg, about 6 mg, about 12 mg, about 25 mg, about 50 mg or about 75 mg.

In a particularly preferred embodiment, the invention provides a pharmaceutical combination comprising pamapimod or a pharmaceutically acceptable salt thereof and pioglitazone or a pharmaceutically acceptable salt thereof (e.g. pioglitazone hydrochloride), wherein the amount of pamapimod or a pharmaceutically acceptable salt thereof in the combination is about 2 mg to about 75 mg, preferably about 4 to about 50 mg, more preferably about 6 to about 25 mg, most preferably about 12 mg or about 25 mg and wherein the amount of pioglitazone or a pharmaceutically acceptable salt thereof in the combination is from about 8 mg to about 12 mg, preferably about 12 mg.

In another particularly preferred embodiment, the invention provides a pharmaceutical combination comprising pamapimod or a pharmaceutically acceptable salt thereof and pioglitazone or a pharmaceutically acceptable salt thereof (e.g. pioglitazone hydrochloride), wherein the amount of pamapimod or a pharmaceutically acceptable salt thereof in the combination is from about 0.1 to about 50 mg, preferably from about 0.1 to about 20 mg, more preferably from about 0.1 to about 10 mg, in particular from about 0.1 to about 5 mg, more particular from about 0.1 to about 1 mg and wherein the amount of pioglitazone or a pharmaceutically acceptable salt thereof in the combination is from about 0.1 to about 10 mg, preferably from about 0.1 to about 5 mg, more preferably from about 0.1 to about 1.5 mg, in particular from about 0.1 to about 1 mg.

In one embodiment, there is provided a pharmaceutical combination comprising:

(a) a PPAR agonist;
(b) a p38 kinase inhibitor; and optionally
(c) one or more pharmaceutically acceptable diluents, excipients or carriers
for use in a method of preventing or treating hearing loss in a subject and/or for use in a method of preventing or inhibiting hair cell degeneration or hair cell death in a subject, wherein said p38 kinase inhibitor is preferably inhibiting P38-alpha, P38-beta, P38-gamma or P38-delta or combinations thereof; more preferably inhibiting P38-alpha and/or P38-beta.

In one embodiment, there is provided a pharmaceutical combination comprising:

(a) a PPAR agonist;
(b) a p38 kinase inhibitor; and optionally
(c) one or more pharmaceutically acceptable diluents, excipients or carriers
for use in a method of preventing or treating hearing loss in a subject and/or for use in a method of preventing or inhibiting hair cell degeneration or hair cell death in a subject, wherein said p38 kinase inhibitor is selected from the group consisting of pamapimod, losmapimod, dilmapimod, AZD7624, ARRY-371797, LY2228820, R9111, PH-797804, BIRB 796, VX-702, VX-745, SB 239063, SB202190, SCIO 469, and BMS 582949 or a pharmaceutically acceptable salt thereof.

In a preferred embodiment, there is provided a pharmaceutical combination comprising:

(a) a PPAR agonist;
(b) pamapimod or a pharmaceutically acceptable salt thereof; and optionally
(c) one or more pharmaceutically acceptable diluents, excipients or carriers
for use in a method of preventing or treating hearing loss in a subject and/or for use in a method of preventing or inhibiting hair cell degeneration or hair cell death in a subject.

In a further embodiment, there is provided a pharmaceutical combination according to the invention, comprising
(a) a PPAR agonist;
(b) a compound of formula I as defined herein; and optionally
(c) one or more pharmaceutically acceptable diluents, excipients or carriers
   for use in a method of preventing or treating hearing loss in a subject and/or for use in a method of preventing or inhibiting hair cell degeneration or hair cell death in a subject.

In a further embodiment, there is provided a pharmaceutical combination comprising
(a) a PPAR agonist;
(b) a compound of formula II as defined herein; and optionally
(c) one or more pharmaceutically acceptable diluents, excipients or carriers for use in a method of preventing or treating hearing loss in a subject and/or for use in a method of preventing or inhibiting hair cell degeneration or hair cell death in a subject.

In a further embodiment, there is provided a pharmaceutical combination comprising
(a) a PPAR gamma agonist;
(b) a compound of formula I as defined herein; and optionally
(c) one or more pharmaceutically acceptable diluents, excipients or carriers
   for use in a method of preventing or treating hearing loss in a subject and/or for use in a method of preventing or inhibiting hair cell degeneration or hair cell death in a subject.

In a further embodiment, there is provided a pharmaceutical combination comprising
(a) a PPAR gamma agonist;
(b) a compound of formula II as defined herein; and optionally
(c) one or more pharmaceutically acceptable diluents, excipients or carriers
   for use in a method of preventing or treating hearing loss in a subject and/or for use in a method of preventing or inhibiting hair cell degeneration or hair cell death in a subject.

In a preferred embodiment, there is provided a pharmaceutical combination comprising
(a) a PPAR gamma agonist;
(b) a compound of formula I as defined herein; and optionally
(c) one or more pharmaceutically acceptable diluents, excipients or carriers
   for use in a method of preventing or treating hearing loss in a subject and/or for use in a method of preventing or inhibiting hair cell degeneration or hair cell death in a subject; wherein said PPAR gamma agonist is selected from the group consisting of pioglitazone, rosiglitazone, troglitazone and INT131 or a pharmaceutically acceptable salt thereof; and
   wherein X¹ and X² in said compound of formula I are each O; and
   wherein Z in said compound of formula I is N; and
   wherein W in said compound of formula I is NH; and
   wherein Ar¹ in said compound of formula I is aryl; and
   wherein R¹ in said compound of formula I is heteroalkyl; and
   wherein R³ in said compound of formula I is alkyl.

In a further preferred embodiment, there is provided a pharmaceutical combination comprising
(a) a PPAR gamma agonist;
(b) pamapimod or a pharmaceutically acceptable salt thereof or R9111 or a pharmaceutically acceptable salt thereof, preferably pamapimod or a pharmaceutically acceptable salt thereof; and optionally
(c) one or more pharmaceutically acceptable diluents, excipients or carriers
   for use in a method of preventing or treating hearing loss in a subject and/or for use in a method of preventing or inhibiting hair cell degeneration or hair cell death in a subject; wherein said PPAR gamma agonist is selected from the group consisting of pioglitazone, rosiglitazone and troglitazone or a pharmaceutically acceptable salt thereof.

In a particularly preferred embodiment, there is provided a pharmaceutical combination comprising:

(a) pioglitazone or a pharmaceutically acceptable salt thereof, preferably pioglitazone hydrochloride;
(b) pamapimod or a pharmaceutically acceptable salt thereof; and optionally
(c) one or more pharmaceutically acceptable diluents, excipients or carriers
for use in a method of preventing or treating hearing loss in a subject and/or for use in a method of preventing or inhibiting hair cell degeneration or hair cell death in a subject.

### Pharmaceutical Compositions

In some embodiments, the pharmaceutical combinations of the invention include other medicinal or pharmaceutical agents, diluents, excipients, carriers, adjuvants, such as preserving, stabilizing, wetting or emulsifying agents, solution promoters, salts for regulating the osmotic pressure, and/or buffers. Diluents are e.g. water, glycols, oils or alcohols. Carriers are e.g. starches or sugars. Excipients are e.g. surface-active substances, emulsifiers, stabilizers, preservatives, flavorings, or fillers.

In other embodiments, the pharmaceutical combinations of the invention also contain other therapeutic substances. Optionally, otoprotective agents, such as antioxidants, alpha lipoic acid, calcium, fosfomycin or iron chelators, to counteract potential ototoxic effects that may arise from the use of specific therapeutic agents or excipients, diluents or carriers are included in the pharmaceutical combinations of the invention.

In some embodiments, the pharmaceutical combinations of the invention include a dye to help enhance the visualization of the pharmaceutical combination when applied. In other embodiments, the pharmaceutical combinations of the invention also include one or more pH adjusting agents or buffering agents to provide an endolymph or perilymph suitable pH. Suitable pH adjusting agents or buffers include, but are not limited to acetate, bicarbonate, ammonium chloride, citrate, phosphate, pharmaceutically acceptable salts thereof or combinations or mixtures thereof. Such pH adjusting agents and buffers are included in an amount required to maintain pH of the composition between a pH of about 5 and about 9, in a preferred embodiment a pH between about 6.5 to about 7.5.

### Modes of Administration and Treatment

Drugs delivered to the inner ear and/or to the middle ear have been administered systemically via oral, intravenous or intramuscular routes. The pharmaceutical combinations used in the methods described herein are usually administered orally, topically in the ear or by injection into the inner ear and/or into the middle ear, preferably by injection into the middle ear. For some routes of administration, e.g. for injection into the inner ear and/or into the middle ear a sustained release sytem can be used. In some routes of administration the penetration of the active ingredient is facilitated by transport enhancers as e.g. hyaluronic acid, DMSO. In some routes of administration, in particular when the pharmaceutical combination of the invention is administered by injection into the inner ear and/or into the middle ear a tixotropic or thermogeling formulation is used to enable a painless administration and forming a gel or a high viscous composition ensuring prolonged and continuous release of the active ingredient into the inner ear and/or into the middle ear. In some routes of administration, in particular when the pharmaceutical combination of the invention is administered as ear drops a formulation that enhances penetration through the skin leading i.a. to local PPAR activation in the ear region can be used.

The pharmaceutical combination of the invention can be located in contact with the crista fenestrae cochlea, the round window, the tympanic cavity, the tympanic membrane, the auris media or the auris externa. In further or alternative embodiments, the pharmaceutical combination of the invention can be administered on or near the round window membrane via intratympanic injection. In other embodiments, the pharmaceutical combination of the invention is administered on or near the round window or the crista fenestrae cochleae through entry via a post-auricular incision and surgical manipulation into or near the round window or the crista fenestrae cochleae area. Alternatively, the pharmaceutical combination of the invention is applied via syringe and needle, wherein the needle is inserted through the tympanic membrane and guided to the area of the round window or crista fenestrae cochleae. The pharmaceutical combination of the invention is then deposited on or near the round window or crista fenestrae cochleae for localized treatment.

Preferably the pharmaceutical combinations as described herein are administered by intratympanic injection into the inner ear and/or into the middle ear, preferably into the middle ear. Intratympanic injection of therapeutic agents is the technique of injecting an agent behind the tympanic membrane into the middle and/or inner ear, preferably into the middle ear.

In one embodiment, the pharmaceutical combinations described herein are administered directly onto the round window membrane via transtympanic injection. In another embodiment, the pharmaceutical combination of the invention is an auris-acceptable pharmaceutical combination and is administered onto the round window membrane via a non-transtympanic approach to the inner ear. In additional embodiments, the pharmaceutical combinations described herein are administered onto the round window membrane via a surgical approach to the round window membrane comprising modification of the crista fenestrae cochleae.

In one embodiment the delivery system is a syringe and needle apparatus that is capable of piercing the tympanic membrane and directly accessing the round window membrane or crista fenestrae cochleae of the auris interna.

In some embodiments, the delivery device is an apparatus designed for administration of therapeutic agents to the middle and/or inner ear. By way of example only: GYRUS Medical Gmbh offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446. U.S. patent application Ser. No. 08/874,208 describes a surgical method for implanting a fluid transfer conduit to deliver therapeutic agents to the inner ear. U.S. Patent Application Publication 2007/0167918 further describes a combined otic aspirator and medication dispenser for intratympanic fluid sampling and medicament application.

The pharmaceutical combinations described herein are useful in surgical procedures including, by way of non-limiting examples, cochlea surgery, labyrinthotomy, mastoidectomy, stapedectomy, endolymphatic sacculotomy or the like. In a preferred embodiment the pharmaceutical combinations as described herein are administered prior to surgical procedures in particular prior to cochlea surgery.

The pharmaceutical combinations described herein are administered for preventive and/or therapeutic treatments. Preventive treatments comprise prophylactic treatments. In preventive applications, the pharmaceutical combination of the invention is administered to a subject suspected of having, or at risk for developing a disease, disorder or condition as described herein. In therapeutic applications, the pharmaceutical combination of the invention is administered to a subject such as a patient already suffering from a disorder disclosed herein, in an amount sufficient to cure or at least partially arrest the symptoms of the disease, disorder or condition as described herein. Amounts effective for this use will depend on the severity and course of the disease, disorder or condition, previous therapy, the subject's health status and response to the drugs, and the judgment of the treating physician.

In the case wherein the subject's condition does not improve, the administration of the pharmaceutical combination of the invention may be administered chronically, which is, for an extended period of time, including throughout the duration of the subject's life in order to ameliorate or otherwise control or limit the symptoms of the subject's disease or condition. In the case wherein the subject's status does improve, the administration of the pharmaceutical combination of the invention may be given continuously; alternatively, the dose of drug being administered may be temporarily reduced or temporarily suspended for a certain length of time (i.e., a “drug holiday”).

Once improvement of the patient's otic conditions has occurred, a maintenance dose of the pharmaceutical combination of the invention is administered if necessary. Subsequently, the dosage or the frequency of administration, or both, is optionally reduced, as a function of the symptoms, to a level at which the improved disease, disorder or condition is retained.

In some preferred embodiments the pharmaceutical combination of the invention is administered by a single injection into the inner ear and/or into the middle ear, preferably by a single intratympanic injection into the inner ear followed by oral administration or by a single intratympanic injection into the middle ear followed by oral administration, which is preferred, or by administration as ear drops with penetration into the inner ear. Oral administration can be provided chronically, which is, for an extended period of time, including throughout the duration of the subject's life. In some embodiments after long term treatment, e.g. long term treatment using oral administration hearing capacity is increased based on a reactivation of hair cells from a resting state and/or improvement in neuronal function. In some embodiments after long term treatment, e.g. long term treatment using oral administration hearing capacity is increased based on an increase of the number of hair cells or hair cell function and/or improvement in neuronal function subsequent to PPAR activation.

The amount of the pharmaceutical combination of the invention to be administered will vary depending upon factors such as the particular compound, disease condition and its severity, according to the particular circumstances surrounding the case, including, e.g., the specific PPAR agonist and the specific p38 inhibitor being administered, the route of administration, the condition being treated, the target area being treated, and the subject or host being treated. In some embodiments the pharmaceutical combination of the invention is administered to the subject in a dose that comprises a dose of a PPAR agonist which is below the dose needed for the treatment of diabetes using a PPAR agonist. In some embodiments the pharmaceutical combination of the invention is administered to the subject in a dose that comprises a dose of a PPAR agonist which is a factor of 8-20 fold lower than the top dose evaluated and tested for the treatment of diabetes, in particular a factor of 8-20 fold lower than the top dose evaluated and tested for the treatment of diabetes in human. The top dose evaluated and tested for the treatment of diabetes in human, e.g for a PPAR gamma agonist such as pioglitazone hydrochloride is usually in the range from about 30-45 mg/day. In some embodiments at the PPAR agonist dose used the side effects seen in treatment of diabetes using said PPAR agonist are not present.

In some embodiments the pharmaceutical combination of the invention is administered to the subject in a dose that comprises a dose of a PPAR agonist which is below the active dose for antidiabetic or anti-dyslipidemic effect of the PPAR agonist, in particular a dose that is below the active dose for antidiabetic or anti-dyslipidemic effect of the the PPAR agonist in human. A typical dosing regimen of pioglitazone or a pharmaceutically acceptable salt thereof in the treatment of diabetes includes 15 to 45 mg pioglitazone once-daily.

In some embodiments, the pharmaceutical combination of the invention is administered orally to a human in a dose comprising a dose of a PPAR agonist, usually PPAR gamma agonists, PPAR alpha agonists and/or PPAR alpha/gamma dual agonists, preferably a PPAR gamma agonist, more preferably pioglitazone or a pharmaceutically acceptable salt thereof, more preferably pioglitazone hydrochloride, most preferably micronized pioglitazone hydrochloride of 0.05-30 mg/day, preferably 0.1-10 mg/day, more preferably 0.5-5 mg/day and/or comprising a dose of a compound of the formula I or II, usually a compound of the formula I, preferably a p38 inhibitor selected from the group consisting of pamapimod, losmapimod, dilmapimod, AZD7624, ARRY-371797, LY2228820, R9111, PH-797804, BIRB 796, VX-702, VX-745, SB 239063, SB202190, SCIO 469, and BMS 582949 or a pharmaceutically acceptable salt thereof, more preferably a p38 kinase inhibitors selected from the group consisting of pamapimod, losmapimod, dilmapimod, ARRY-371797, LY2228820, R9111, PH-797804, BIRB 796, VX-702, VX-745, SB 239063, SB202190, SCIO 469, and BMS 582949 or a pharmaceutically acceptable salt thereof, even more preferably pamapimod in particular micronized pamapimod or a pharmaceutically acceptable salt thereof of 1-500 mg/day, preferably 10-250 mg/day, more preferably 25-150 mg/day.

In some embodiments, the pharmaceutical combination of the invention is administered orally to a human in a dose comprising a dose of a PPAR agonist, usually PPAR gamma agonists, PPAR alpha agonists and/or PPAR alpha/gamma dual agonists, preferably a PPAR gamma agonist, more preferably pioglitazone or a pharmaceutically acceptable salt thereof, more preferably pioglitazone hydrochloride, most preferably micronized pioglitazone hydrochloride of 0.05-30 mg/day, preferably 0.1-10 mg/day, more preferably 0.1-1.5 mg/day, in particular 0.1-1 mg/day and/or comprising a dose of a compound of the formula I or II, usually a compound of the formula I, preferably a p38 inhibitor selected from the group consisting of pamapimod, losmapimod, dilmapimod, AZD7624, ARRY-371797, LY2228820, R9111, PH-797804, BIRB 796, VX-702, VX-745, SB 239063, SB202190, SCIO 469, and BMS 582949 or a pharmaceutically acceptable salt thereof, more preferably a p38 kinase inhibitors selected from the group consisting of pamapimod, losmapimod, dilmapimod, ARRY-371797, LY2228820, R9111, PH-797804, BIRB 796, VX-702, VX-745, SB 239063, SB202190, SCIO 469, and BMS 582949 or a pharmaceutically acceptable salt thereof, even more preferably pamapimod in particular micronized pamapimod or a pharmaceutically acceptable salt thereof of 0.05-100 mg/day, preferably 0.1-50 mg/day, more preferably 0.1-15 mg/day, in particular 0.1-5 mg/day.

In some embodiments, the pharmaceutical combination of the invention is administered to a human topically in the ear usually comprising the PPAR agonist, usually PPAR gamma agonists, PPAR alpha agonists and/or PPAR alpha/gamma dual agonists, preferably a PPAR gamma agonist, more preferably pioglitazone or a pharmaceutically acceptable salt thereof, more preferably pioglitazone hydrochloride, most preferably micronized pioglitazone hydrochloride at a concentration of 0.001% w/v to 10% w/v, preferably at a concentration of 0.005% w/v to 5% w/v, more preferably at a concentration of 0.01% w/v to 2% w/v and/or comprising the compound of the formula I or II, usually a compound of the formula I, preferably a p38 inhibitor selected from the group consisting of pamapimod, losmapimod, dilmapimod, AZD7624, ARRY-371797, LY2228820, R9111, PH-797804, BIRB 796, VX-702, VX-745 SB, 239063, SB202190, SCIO 469, and BMS 582949 or a pharmaceutically acceptable salt thereof, more preferably a p38 kinase inhibitors selected from the group consisting of pamapimod, losmapimod, dilmapimod, ARRY-371797, LY2228820, R9111, PH-797804, BIRB 796, VX-702, VX-745, SB 239063, SB202190, SCIO 469, and BMS 582949 or a pharmaceutically acceptable salt thereof, even more preferably pamapimod, in particular micronized pamapimod or a pharmaceutically acceptable salt thereof at a concentration of of 0.001% w/v to 20% w/v, preferably at a concentration of 0.01% w/v to 15% w/v, more preferably at a concentration of 0.02% w/v to 10% w/v.

In a preferred embodiment, the pharmaceutical combination of the invention is a solution and usually 50 μl to 1 ml, preferably 1 ml of said solution is administered topically in the ear.

In some embodiments, the pharmaceutical combination of the invention is administered to a human by injection into the inner ear and/or into the middle ear comprising the PPAR agonist, usually PPAR gamma agonists, PPAR alpha agonists and/or PPAR alpha/gamma dual agonists, preferably a PPAR gamma agonist, more preferably pioglitazone or a pharmaceutically acceptable salt thereof, more preferably pioglitazone hydrochloride, most preferably micronized pioglitazone hydrochloride at a concentration of about 0.01% to about 7.5% w/v, preferably about 0.01% to about 5% w/v, more preferably about 0.1% to about 7.5% w/v, more preferably about 0.1% to about 5% w/v, more preferably about 0.1% to about 4% w/v, more preferably about 0.1% to about 3% w/v, more preferably about 0.1% to about 2% w/v, more preferably about 0.5% to about 2% w/v, more preferably about 0.5% to about 1.5% w/v, more preferably about 1% to about 1.5% w/v, most preferably about 1.2% w/v per single injection and/or comprising a compound of the formula I or II, usually a compound of the formula I, preferably a p38 inhibitor selected from the group consisting of pamapimod, losmapimod, dilmapimod, AZD7624, ARRY-371797, LY2228820, R9111, PH-797804, BIRB 796, VX-702, VX-745, SB 239063, SB202190, SCIO 469, and BMS 582949 or a pharmaceutically acceptable salt thereof, more preferably a p38 kinase inhibitors selected from the group consisting of pamapimod, losmapimod, dilmapimod, ARRY-371797, LY2228820, R9111, PH-797804, BIRB 796, VX-702, VX-745, SB 239063, SB202190, SCIO 469, and BMS 582949 or a pharmaceutically acceptable salt thereof, even more preferably pamapimod in particular micronized pamapimod or a pharmaceutically acceptable salt thereof at a concentration of about 0.01% to about 20% w/v, preferably about 0.01% to about 17.5% w/v, more preferably about 0.02% to about 15% w/v, more preferably about 0.02% to about 10% w/v, more preferably about 0.1% to about 4% w/v, more preferably about 0.1% to about 3% w/v, more preferably about 0.1% to about 2% w/v, more preferably about 0.5% to about 2% w/v, more preferably about 0.5% to about 1.5% w/v, more preferably about 1% to about 1.5% w/v, most preferably about 1.2% w/v per single injection, preferably per single intratympanic injection.

In a preferred embodiment, the pharmaceutical combination of the invention is a solution and usually 50 μl to 1 ml, preferably 1 ml of said solution is injected by a single injection.

Methods of identification of patients who are suspected of having, or being at risk for developing hearing loss, hair cell degeneration or hair cell death are also comprised by the present invention. In some embodiments patients who are suspected of having, or being at risk for developing hearing loss, hair cell degeneration or hair cell death are identified by measurement of serum and/or plasma adiponectin levels, in particular the measurement of high molecular weight adiponectin levels. In some embodiments the monitoring of the treatment success and/or the identification of the subject e.g. the identification of the subject who is suspected of having, or being at risk for developing hearing loss, hair cell degeneration or hair cell death, is achieved by measurement of serum and/or plasma adiponectin levels.

### Kits/Articles of Manufacture

The disclosure also provides kits for preventing or treating hearing loss and/or for preventing or inhibiting hair cell degeneration or hair cell death in a subject, preferably in human. Such kits generally will comprise one or more pharmaceutical combination disclosed herein, and instructions for using the kit.

In a preferred embodiment a kit for preventing or treating hearing loss and/or for preventing or inhibiting hair cell degeneration or hair cell death in a subject, comprises a pharmaceutical combination comprising:

(a) a PPAR agonist;
(b) a p38 kinase inhibitor; and optionally
(c) one or more pharmaceutically acceptable diluents, excipients or carriers;
and instructions for using the kit.

Preferred PPAR agonist and preferred p38 kinase inhibitor are as described above. The disclosure also contemplates the use of one or more pharmaceutical combination disclosed herein, in the manufacture of medicaments for treating, abating, reducing, or ameliorating the symptoms of a disease, dysfunction, or disorder in a mammal, such as a human that has, is suspected of having, or at risk for developing hearing loss, hair cell degeneration or hair cell death.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

The articles of manufacture provided herein generally will comprise one or more pharmaceutical combination disclosed herein and packaging materials. Examples of pharmaceutical packaging materials include, but are not limited to, blister packs, bottles, tubes, inhalers, pumps, bags, vials, containers, syringes, bottles, and any packaging material suitable for a selected composition and intended mode of administration and treatment.

### EXAMPLES

### Example 1: Additive Protection Against Antibiotic-Induced Hair Cell Loss Conferred by the Combination of a PPAR Agonist and P38 Inhibitor

Organs of Corti were obtained from post-natal day 5 C57bl/6j mice and placed in organ culture. Gentamicin treatment resulted in 50% loss of hair cells after 24 hr. in culture. Treatment with the single agents (5 μM pioglitazone (micronized, Dv50=4 μm) or 30 μM pamapimod (micronized, Dv50=3 μm)) partially prevented the loss of hair cells. A combination of both agents (5 μM pioglitazone (micronized, Dv50=4 μm) and 30 μM pamapimod (micronized, Dv50=3 μm)) was fully protective, demonstrating the additive effects of both agents.

### Methods

### Animal Procedures

All animal procedures were carried out according to protocols approved by the Kantonales Veterinäramt, Basel, Switzerland. Postnatal day 5 (p5) C57bl/6j were used for the studies. Studies were performed in vitro, using organ of Corti (OC) explants from p5 animals. Animals were sacrificed and the cochleae carefully dissected to separate the organ of Corti from the spiral ganglion, stria vascularis and Reissner's membrane [Sobkowicz H M, Loftus J M, Slapnick S M. Acta Otolaryngol Suppl. 1993; 502:3-36].

### Tissue Culture

OCs were harvested and then placed in culture medium [Dulbecco's Modified Eagle Medium supplemented with 10% FCS, 25 mM HEPES and 30 U/ml penicillin (Invitrogen, Carlsbad, 20 CA, USA)] and incubated for 24 hours at 37° C. in an atmosphere of 95% O₂/5% CO₂. After that period, the culture medium was replaced with fresh medium containing no compound or 50 μM gentamicin alone or 50 μM gentamicin with either 5 μM pioglitazone, 30 μM pamapimod, or 5 μM pioglitazone+30 μM pamapimod, and incubated for a further 24 hours at 37° C. Ten OC explants were used for each treatment condition.

### Auditory Hair Cell Counting

After incubation with compounds, the OCs were fixed in 4% paraformaldehyde, washed and then stained with a fluorescein (FITC)-conjugated phalloidin to detect inner and outer hair cells. After staining, the OCs were visualized and photographed using a fluorescence microscope (Olympus FSX100). Auditory hair cells were quantified for the basal turn of each organ of Corti in 3 fields corresponding to 80 hair cells each. The average values for the three fields were then averaged for the 10 OCs used for each condition. Significant differences between treatment groups in numbers of hair cells were determined using analysis of variance (ANOVA) followed by the least significant difference (LSD) post-hoc test (Stat View 5.0). Differences associated with P values of less than 0.05 were considered to be statistically significant. All data are presented as mean±SD.

### Results

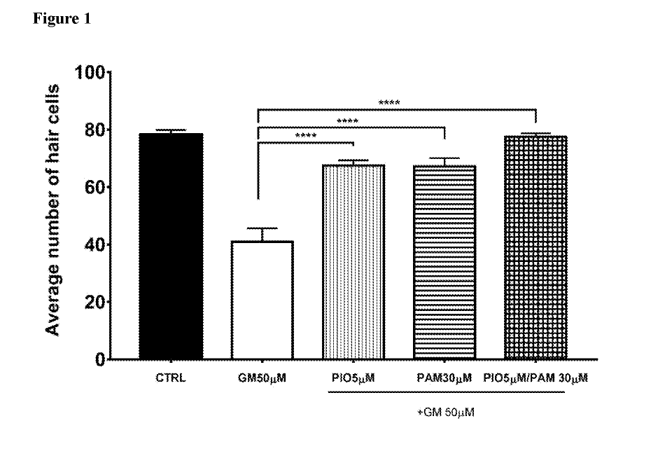

Untreated organs of Corti were well preserved after 48 hours in culture presenting with intact ordered rows of outer hair cells (OHC) and inner hair cells (IHC). Treatment with 50 μM gentamicin resulted in approximately 50% loss of hair cells (FIG. 1). Pioglitazone at 5 μM or pamapimod at 30 μM partially protected hair cells. In each condition, only approximately 20% of hair cells were lost. In contrast, the combination of pioglitazone at 5 μM+pamapimod at 30 μM resulted in preservation of 100% of the hair cells.

### Example 2: Synergistic Protection Against Antibiotic-Induced Hair Cell Loss Conferred by the Combination of a PPAR Agonist and P38 Inhibitor

A similar experiment was performed as in Example 1 to assess the effects of lower combined concentrations of the PPAR agonist pioglitazone with the P38 inhibitor pamapimod on auditory hair cell protection from gentamicin toxicity. Organs of Corti were obtained from post-natal day 5 C57bl/6j mice and placed in organ culture. Gentamicin treatment resulted in 50% loss of hair cells after 24 hr. in culture. Treatment with the single agents (1 μM pioglitazone or 1 μM pamapimod) partially prevented the loss of hair cells with pioglitazone providing approximately 40% protection and pamapimod providing approximately 30% protection. Surprisingly and unexpectedly, the combination of 1 μM pioglitazone and 1 μM pamapimod provided approximately 100% protection, demonstrating synergy in improving efficacy beyond simply the additive effects of each agent at these concentrations alone.

### Methods

### Animal Procedures

All animal procedures were carried out according to protocols approved by the Kantonales Veterinäramt, Basel, Switzerland. Postnatal day 5 (p5) C57bl/6j were used for the studies. Studies were performed in vitro, using organ of Corti (OC) explants from p5 animals. Animals were sacrificed and the cochleae carefully dissected to separate the organ of Corti from the spiral ganglion, stria vascularis and Reissner's membrane [Sobkowicz H M, Loftus J M, Slapnick S M. Acta Otolaryngol Suppl. 1993; 502:3-36].

### Tissue Culture

OCs were harvested and then placed in culture medium [Dulbecco's Modified Eagle Medium supplemented with 10% FCS, 25 mM HEPES and 30 U/ml penicillin (Invitrogen, Carlsbad, 20 CA, USA)] and incubated for 24 hours at 37° C. in an atmosphere of 95% O₂/5% CO₂. After that period, the culture medium was replaced with fresh medium containing no compound or 100 M gentamicin alone or 100 μM gentamicin with either 1 μM pioglitazone, 1 μM pamapimod, or 1 μM pioglitazone+1 μM pamapimod, and incubated for a further 24 hours at 37° C. The gentamicin concentration of 100 μM was determined as that concentration that leads to 50% loss of hair cells. Batch to batch variation in the activity of gentamicin requires that the concentration used for specific experiments leading to 50% hair cell loss be predefined in pilot experiments for each batch. Ten OC explants were used for each treatment condition.

### Auditory Hair Cell Counting

After incubation with compounds, the OCs were fixed in 4% paraformaldehyde, washed and then stained with a fluorescein (FITC)-conjugated phalloidin to detect inner and outer hair cells. After staining, the OCs were visualized and photographed using a fluorescence microscope (Olympus FSX100). Auditory hair cells were quantified for the basal turn of each organ of Corti in 3 fields corresponding to 80 hair cells each. The average values for the three fields were then averaged for the 10 OCs used for each condition. Significant differences between treatment groups in numbers of hair cells were determined using analysis of variance (ANOVA) followed by the least significant difference (LSD) post-hoc test (Stat View 5.0). Differences associated with P values of less than 0.05 were considered to be statistically significant. All data are presented as mean±SD.

### Results

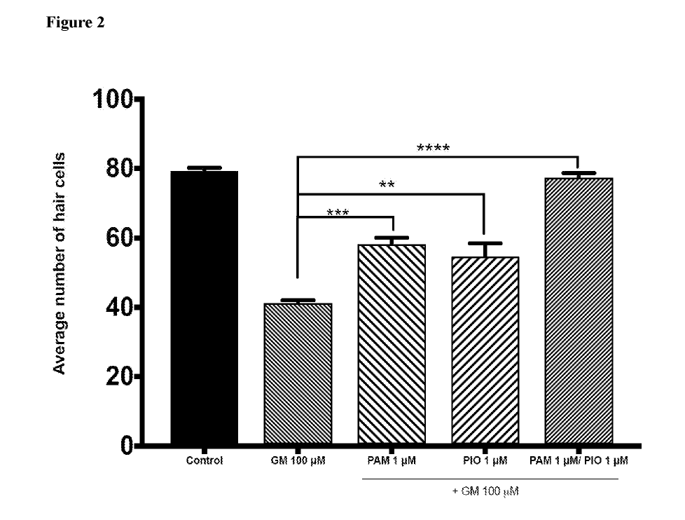

Untreated organs of Corti were well preserved after 48 hours in culture presenting with intact ordered rows of outer hair cells (OHC) and inner hair cells (IHC). Treatment with 100 μM gentamicin resulted in approximately 50% loss of hair cells (FIG. 2). Treatment with the single agents (1 μM pioglitazone or 1 μM pamapimod) partially prevented the loss of hair cells with pioglitazone providing approximately 40% protection and pamapimod providing approximately 30% protection. Surprisingly and unexpectedly, the combination of 1 μM pioglitazone and 1 μM pamapimod provided approximately 100% protection, demonstrating a synergistic effect.

1. A method of preventing hearing loss, hair cell degeneration, or hair cell death, treating hearing loss, or inhibiting hair cell degeneration or hair cell death in a subject, comprising administering a pharmaceutical combination to the subject in need thereof, wherein the pharmaceutical combination:
(a) a PPAR agonist;
(b) a p38 kinase inhibitor; and optionally
(c) one or more pharmaceutically acceptable diluents, excipients or carriers.

2. The method of claim 1, wherein the p38 kinase inhibitor is a compound of formula I or II 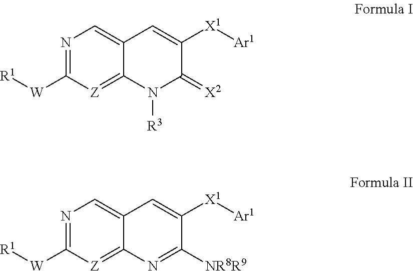
or pharmaceutically acceptable salts thereof, wherein
Z is N or CH;
W is NR²;
X¹ is O, NR⁴ (where R⁴ is hydrogen or alkyl), S, or CR⁵R⁶ (where R⁵ and R⁶ are independently hydrogen or alkyl) or C═O;
X² is O or NR⁷;
Ar¹ is aryl or heteroaryl;
R² is hydrogen, alkyl, acyl, alkoxycarbonyl, aryloxycarbonyl, heteroalkylcarbonyl, heteroalkyloxycarbonyl or —R²¹-R²² where R²¹ is alkylene or —C(═O)— and R²² is alkyl or alkoxy;
R¹ is hydrogen, alkyl, haloalkyl, aryl, aralkyl, heteroaryl, heteroaralkyl, cycloalkyl, cycloalkylalkyl, heteroalkyl substituted cycloalkyl, heterosubstituted cycloalkyl, heteroalkyl, cyanoalkyl, heterocyclyl, heterocyclylalkyl, R¹²—SO₂-heterocycloamino (where R¹² is haloalkyl, aryl, aralkyl, heteroaryl or heteroaralkyl), —Y¹—C(O)—Y²—R¹¹ (where Y¹ and Y² are independently either absent or an alkylene group and R¹¹ is hydrogen, alkyl, haloalkyl, hydroxy, alkoxy, amino, monoalkylamino or dialkylamino), (heterocyclyl)(cycloalkyl)alkyl or (heterocyclyl)(heteroaryl)alkyl;
R³ is hydrogen, alkyl, cycloalkyl, cycloalkylalkyl, aryl, aralkyl, haloalkyl, heteroalkyl, cyanoalkyl, alkylene-C(O)—R³¹ (where R³¹ is hydrogen, alkyl, hydroxy, alkoxy, amino, monoalkylamino or dialkylamino), amino, monoalkylamino, dialkylamino or NR³²—Y³—R³³ (where Y³ is —C(O), —C(O)O—, —C(O)NR³⁴, S(O)₂ or S(O)₂NR³⁵; R³², R³⁴ and R³⁵ are independently hydrogen or alkyl; and R³³ is hydrogen, alkyl, cycloalkyl, cycloalkylalkyl, heteroalkyl or optionally substituted phenyl) or acyl;
R⁷ is hydrogen or alkyl; and
R⁸ and R⁹ are independently hydrogen, alkyl, aryl, aralkyl, cycloalkyl, cycloalkylalkyl, heteroalkyl, alkylsulfonyl, arylsulfonyl, —C(O)—R⁸¹ (where R⁸¹ is alkyl, aryl, aralkyl, cycloalkyl, cycloalkylalkyl, heteroalkyl, alkoxy, aryloxy, amino, mono- or di-alkylamino, arylamino or aryl(alkyl)amino) or R⁸ and R⁹ together form ═CR⁸²R⁸³ (where R⁸² and R⁸³ are independently hydrogen, alkyl, cycloalkyl, cycloalkylalkyl or optionally substituted phenyl).

3. The method of claim 2, wherein the p38 kinase inhibitor is a compound of formula I or a pharmaceutically acceptable salt thereof.

4. The method of claim 3, wherein said compound of formula I is 6-(2,4-Difluorophenoxy)-2-[3-hydroxy-1-(2-hydroxyethyl)-propylamino]-8-methyl-8H-pyrido[2,3-d]pyrimidin-7-one (pamapimod, Formula III) or a pharmaceutically acceptable salt thereof.

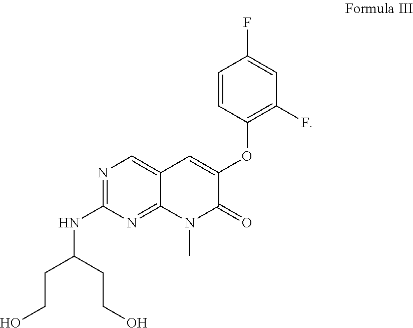 [SMILES: Cn1c(=O)c(Oc2ccc(F)cc2F)cc2cnc(NC(CCO)CCO)nc21]

5. The method of claim 1, wherein the PPAR agonist is pioglitazone or a pharmaceutically acceptable salt thereof.

6. The method of claim 1, wherein hearing loss, hair cell degeneration or hair cell death is caused by hair cell damage.

7. The method of claim 1, wherein hearing loss is sudden hearing loss.

8. The method of claim 1, wherein hearing loss is caused by a noise trauma, by a medical intervention, by ischemic injury, by age or is chemically induced.

9. The method of claim 1, wherein the combination is administered prior to cochlea surgery.

10. The method of claim 1, wherein the combination is administered orally, topically in the ear, by injection into the inner ear and/or by injection into the middle ear.

11. The method of claim 1, wherein the combination is administered by intratympanic injection into the middle ear.

12. The method of claim 1, wherein the combination is administered by a single injection into the inner ear and/or into the middle ear followed by oral administration or followed by administration as ear drops with penetration into the inner ear.

13. The method of claim 1, wherein the combination is administered to the subject in a dose that comprises a dose of a PPAR agonist which is below the dose needed for the treatment of diabetes using said PPAR agonist.

14. The method of claim 2, wherein the subject is a human and the combination is administered orally in a dose corresponding to 0.5-5 mg/day of a PPAR agonist and/or in a dose corresponding to 25-150 mg/day of the compound of formula I or II, topically in the ear in a dose corresponding to 0.01% to 2% of a PPAR agonist and/or in a dose corresponding 0.02% to 10% of the compound of formula I or II or by injection into the inner ear and/or into the middle ear at a concentration of 0.01% to 5% wt/wt PPAR agonist and/or at a concentration of 0.02% to 10% wt/wt of the compound of formula I or II per single injection.

15. A kit for preventing or treating hearing loss and/or for preventing or inhibiting hair cell degeneration or hair cell death in a subject, comprising a pharmaceutical combination comprising:
(a) a PPAR agonist;
(b) a p38 kinase inhibitor; and optionally
(c) one or more pharmaceutically acceptable diluents, excipients or carriers;
and instructions for using the kit.
